(19) United States
(12) Patent Application Publication
(10) Pub. No.: US 2021/0186927 A1
(43) Pub. Date: Jun. 24, 2021

(54) COMBINATION PRODUCT FOR THE INDUCTION AND/OR MAINTENANCE OF GENERAL ANESTHESIA

(71) Applicants: BLUMENTECH, S.L., BARCELONA (ES); INSTITUT DE RECERCA DE L'HOSPITAL DE LA SANTA CREU I SANT PAU, BARCELONA (ES)

(72) Inventor: Jordi RIBA SERRANO, BARCELONA (ES)

(21) Appl. No.: 17/047,018

(22) PCT Filed: Apr. 12, 2019
(86) PCT No.: PCT/EP2019/059380

§ 371 Date: Oct. 12, 2020

(30) Foreign Application Priority Data
Apr. 13, 2018 (EP) 18382252.7

(51) Int. Cl. A61K 31/366; A61K 31/4174; A61K 31/5513; A61P 23/00

(52) U.S. Cl. CPC A61K 31/366; A61P 23/00; A61K 31/5513; A61K 31/4174

(57) ABSTRACT

The state of general anesthesia (GA) is essential to many surgical and medical procedures. This state is characterized by loss of consciousness, deep analgesia and suppression of movements. GA is rarely achieved with a single drug, usually requiring the combination of various pharmacological agents. Each drug can interact with one or more molecular targets affecting neuronal excitability and synaptic transmission in multiple regions of the CNS. Agonists of the μ-opioid receptor are commonly used in GA to cause analgesia, but not to induce or maintain loss of consciousness or movement suppression. Additionally, agonists of the μ-opioid receptor can cause serious unwanted side effects, e.g. respiratory depression. The present invention provides alternative combination products based on K-opioid receptor agonists. These combination products unexpectedly induced loss of consciousness, and were able to achieve and maintain GA. Furthermore, the combination products suppressed pain perception without the need of a μ-opioid receptor agonist. The combination of Salvinorin A, a selective κ-opioid receptor agonist, with Diazepam or Medetomidine surprisingly led to rapid consciousness, deep analgesia and movement suppression. This combination was found to effectively induce and maintain a state of general anesthesia.

### TECHNICAL FIELD

The present invention relates to a novel pharmaceutical combination product. The combination product and its components may be used as a medicament, in particular, as a medicament for the induction and/or maintenance of anesthesia.

### BACKGROUND ART

Many surgical procedures, diagnostic tests and therapeutic interventions, in both human and veterinary medicine, can only be conducted under general anesthesia, a reversible state characterized by a deep depression of the central nervous system (CNS). General anesthesia is formally defined as a drug-induced loss of consciousness during which patients cannot be aroused, even by painful stimulation. General anesthesia can be distinguished from deep sedation/analgesia. In the latter state the CNS is also depressed and patients cannot be easily aroused, but nevertheless respond to repeated or painful stimulation (Fish et al., 2011; American Society of Anesthesiologists, 2014).

The state of general anesthesia (GA) is rarely achieved with a single drug, usually requiring the combination of various pharmacological agents. Each drug can interact with one or more molecular targets affecting neuronal excitability and synaptic transmission typically in multiple regions of the CNS (Crowder C M et al., 2013). The criteria that must be fulfilled in order to claim that a state of general anesthesia has been achieved by a drug or drug combination consists of the following:

a) Loss of consciousness.

b) Deep analgesia. Patients cannot be aroused, even by painful stimulation.

c) Suppression of voluntary movements and reflexes.

Prior to the administration of unconsciousness-inducing agents, pre-anesthetic medications may be administered in order to reduce anxiety, produce sedation, and, in veterinary medicine, to facilitate animal manipulation (Muir W W et al., 2013). The most commonly used drugs in humans are positive effectors at the GABA_{A} receptors, such as the benzodiazepines diazepam and midazolam, that act as positive allosteric modulators at this site (Hata T M and Hata J S, 2013). In veterinary medicine, α₂-adrenergic agonists like medetomidine are also used (Muir W W et al., 2013).

For the purpose of the induction and maintenance of the unconscious state, inhaled and intravenous anesthetics may be administered. Many of these compounds also interact with the GABA_{A} receptor. Inhaled anesthetics include gases like nitrous oxide and xenon, and volatile halogenated alkanes like halothane, isoflurane and sevoflurane among others (Ebert T J and Lindenbaum L, 2013). Intravenous anesthetics include barbiturates, propofol, benzodiazepines, etomidate and ketamine, the latter a non-GABA_{A} effector (White P F and Eng M R, 2013). Most inhaled and intravenous anesthetics lack pain-suppressing properties. For this reason, they are frequently associated with opioid agonists, a drug class that displays potent analgesic effects (Dahan A et al., 2013). It is a common practice to co-administer a potent opioid like fentanyl or remifentanil with unconsciousness-inducing drugs in intravenous anesthesia. On the other hand, the use of opioids alone in general anesthesia is rare. Findings indicate that they are not reliable for this purpose and may lead to dangerous respiratory depression (Bailey et al., 1985).

Opioid agonists interact with three main subgroups of opioid receptors. These are G-protein-coupled receptors located on the cellular membranes of neurons and denominated, respectively, μ-opioid receptors (MOR), δ-opioid receptors (DOR), and κ-opioid receptors (KOR) (Waldhoer et al., 2004). The effects of most opioid drugs used in the context of anesthesia rely on their agonist activity at the MOR (Dahan A et al., 2013). MOR agonists used in human and veterinary anesthesia include: natural compounds like morphine; semi-synthetic derivatives like hydromorphone and oxymorphone; and synthetic drugs like meperidine, methadone, fentanyl, remifentanil and alfentanil. MOR agonists are powerful and useful analgesics, but MOR activation can also cause serious side effects. MOR analgesics can potentially induce life-threatening respiratory depression, bradycardia and hypotension. Other adverse effects include nausea, muscle spasms, histamine release, itching, miosis, dizziness, constipation and immunosuppression. Additionally, activation of the MOR induces the reward parthway, i.e., the euphoria and pleasure, associated with opioids drugs, a characteristic that makes MOR agonists prone to being abused and causing physiological dependence and severe addiction (Dahan A et al., 2013).

The “agonist-antagonist” class of opioid drugs has been developed in an attempt to avoid the disadvantages associated with MOR agonists. The agonist-antagonist group includes morphinans butorphanol and nalbuphine, and benzomorphan pentazocine, which display agonist activity at the KOR. The KOR is present in high levels in the CNS (encephalon and spinal cord), also mediates analgesic effects and, most importantly, its activation does not cause respiratory depression and the induction of the reward pathway as with the activation of MOR (Waldhoer et al., 2004). Butorphanol, nalbuphine and pentazocine are used in veterinary anesthesia (Muir W W et al., 2013), whereas butorphanol and nalbuphine are used in obstetrical anesthesia in humans (Braveman F R et al., 2013). While their main analgesic effect is caused by KOR agonism, they are not selective for this receptor. Unfortunately, they also bind to the MOR where they display antagonist or weak partial agonist activity (Waldhoer et al., 2004). This has the important disadvantage of counteracting the effects of full agonists if these are used concomitantly. This interaction can lead to the precipitation of a life-threatening withdrawal syndrome in illicit opioid users (e.g. heroin and/or oxycodone addicts) and also in individuals taking medically-prescribed opioid analgesics (Macres S M et al., 2013). In veterinary medicine, the use of agonist-antagonists in anesthesia impedes the concomitant use of MOR agonists if additional pain suppression is needed, since the former will reduce or completely block the effects of the latter (Muir W W et al., 2013).

To summarize, the primary application of opioid drugs in the context of general anesthesia is pain management. While opioids like fentanyl and remifentanil are commonly co-administered in the induction phase, opioids need to be associated with an unconsciousness-inducing agent. Although MOR agonists are effective analgesics, they are not adequate to achieve or maintain unconsciousness or general anesthesia on their own, even after a preanesthetic drug such as diazepam. In the specific case of the aforementioned agonist-antagonists with KOR activity, they are not used either to achieve or maintain unconsciousness. Analogously to MOR agonists, they are used to treat pain, and even this application is hampered by their antagonistic effects at the MOR. This is due to the lack of selective affinity for the KOR that characterizes the currently available drugs with morphinan and benzomorphan structure.

In recent years, a novel family of selective KOR agonists has been developed. The lead compound is Salvinorin A (SA), a natural substance that can be obtained from the leaves of the plant Salvia divinorum (Labiatae) (Ortega et al., 1982; Valdes et al., 1984). This compound is structurally unrelated to the classic agonist-antagonists with KOR activity discussed above. The SA molecule is a non-nitrogenous terpene with high selectivity, binding almost exclusively to the KOR, where it acts as a full agonist. Its affinity and potency values at this receptor are in the nanomolar range (Roth et al., 2002). Importantly, SA shows no affinity for the MOR, the DOR or any other major CNS receptor class (Roth et al., 2002; Ray, 2010). The discovery of SA has led to the synthesis of a whole new series of highly selective KOR agonists by structural modification of the lead compound. These new substances have the advantage of inducing selective KOR activation, and thus being devoid of the MOR-related side effects typical of the older agonist-antagonist morphinans and benzomorphans.

The potential use of SA and related compounds as unconsciousness-inducing agents in general anesthesia (GA) or as facilitators of the induction and/or maintenance stages of GA has not been tested and cannot be concluded from the literature. Behavioral studies in animals involving the administration of these drugs have yielded inconsistent findings as to their effects on the CNS. Only a few studies have reported sedation, but none have described loss of consciousness after SA. For instance, at 0.4 and 0.64 mg/kg subcutaneously, SA had no effects on the locomotor activity of rats (Beerepoot et al., 2008; Braida et al., 2011). In another study, a single 2 mg/kg administered intraperitoneally (i.p.) had no effect on locomotor activity, whereas repeated daily injections of the same dose actually increased locomotor activity in test animals (Chartoff et al., 2008). No significant changes in response rates were seen in a drug discrimination study involving 1.0-3.0 mg/kg i.p. doses of SA to rats (Willmore-Fordham et al., 2007). On the other hand, as mentioned above, mild sedation and a loss of coordination was observed in mice after 0.5-2 mg/kg i.p. injections of SA (Fantegrossi et al., 2005). In one study conducted in Rhesus monkeys, 0.032 mg/kg administered subcutaneously (s.c.) did not lead to overt sedation (Butelman et al., 2004). However, in a subsequent study the same group reported sedative effects at 0.032 mg/kg intravenously (i.v.) (Butelman et al., 2009).

Rather than regarding SA as a sedative and/or an unconsciousness-inducing drug, the behavioral manifestations it induces in animals have been interpreted as reflecting a state analogous to clinical depression in humans. For instance, SA administration to rats at 0.25-2 mg/kg i.p., increased immobility in the forced swimming test (an animal correlate of depression), but did not affect spontaneous locomotor activity (Carlezon et al., 2006). Based on these findings, SA administration to animals has been proposed as a pre-clinical model to screen for drugs with potential antidepressant activity (Béguin et al., 2008).

Regarding analgesia, one study found dose-dependent antinociceptive effects in the 0.5-4 mg/kg dose range in mice that were administered intraperitoneally (McCurdy et al., 2006), while another reported no effects at 10 mg/kg i.p. in rats (Wang et al., 2008). In a third study involving mice, a 5 mg/kg i.p dose showed no analgesic effects, while 7.5 μg injected intracerebroventricularly were found to be active (Ansonoff et al., 2006).

None of the studies in animals mentioned above suggested that SA alone or in combination with another drug could be used to induce loss of consciousness, or to induce or maintain a state of GA.

Research in humans has also failed to find evidence supporting the use of SA in general anesthesia. Studies in healthy humans have shown that rather than an anesthetic effect, SA brings about an intense hallucinatory state. Despite being structurally unrelated, SA is a powerful hallucinogen like better known drugs such as LSD and psilocybin. The acute administration of SA to humans induces intense visual and auditory hallucinations without loss of consciousness (Maqueda et al., 2015, 2016). These effects are already noticeable at 0.25 mg and perceptual modifications increase with the dose. However, no significant dose-dependent increases are observed in classical psychometric measurements of sedation, such as the PCAG subscale of the Addiction Research Center Inventory (Maqueda et al., 2015). Prior to the administration of doses as high as 1 mg, subjects have to be reminded to remain still, since they can produce voluntary movements, and consciousness is not lost during the acute effects (Maqueda et al., 2015). Consciousness during the acute experience is further evidenced by the fact that individuals are able to provide detailed accounts of the hallucinatory state induced by SA, once the drug's effects have worn off (Maqueda et al., 2015).

The above studies conducted in humans, also show that while perception is altered, drug administration does not lead to the state of unconsciousness that is characteristic of general anesthesia. These findings indicate that SA administered alone is not sufficient to render it useful for the induction or maintenance of general anesthesia. Although similarities between the effects of SA and those of the anesthetic NDMA receptor antagonist ketamine have been pointed out (Siebert. D, 2012), a drug-discrimination study in monkeys did not find that any generalization between the two drugs is applicable (Killinger et al., 2010). Finally, the development of SA into a medicament for use in the context of general anesthesia has not been proposed in the patent literature.

Given the above, there is a need for a combination product that could effectively be used in the induction and/or maintenance of GA. This combination product should: 1) not require the administration of MOR and/or MOR/KOR agonist-antagonists to induce and/or maintain GA so that the combination product can, potentially, be free of the undesired effects associated with MOR agonists (e.g. respiratory suppression, addiction) and with MOR/KOR agonist-antagonists (e.g. induction of withdrawal symptoms in subjects with MOR-agonist dependence); 2) lead to the loss of consciousness, analgesia, and suppression of voluntary movements and reflexes that defines GA; and/or 3) not interfere (antagonize) with MOR agonists or mixed agonist-antagonists, if these drugs need to be administered for additional pain suppression at some stage during GA or thereafter.

### SUMMARY OF THE INVENTION

Given that KOR agonists do not induce respiratory depression and other severe side effects typically displayed by fentanyl, remifentanil and other MOR agonists co-administered during GA induction and maintenance, a medicament containing a selective KOR agonist could be of great advantage. The inventors have found that combinations of KOR with other drugs can, surprisingly, induce general anesthesia. Thus, the present invention provides a combination product comprising (i) one or more selective κ-opioid receptor agonists and (ii) one or more α₂-adrenergic receptor agonists and/or one or more positive GABA_{A} receptor effectors. Further, the present invention provides a pharmaceutical composition comprising the combination product of the present invention, and a pharmaceutically acceptable carrier, a pharmaceutically acceptable diluent and/or a pharmaceutically acceptable excipient.

The present invention also provides the combination product of the present invention and the pharmaceutical composition of the present invention for use as medicament. In a further aspect, the combination product of the present invention and the pharmaceutical composition of the present invention are used to induce and/or maintain general anesthesia in a subject or animal.

The present invention also provides a kit comprising (i) the combination product of the present invention and (ii) a pharmaceutically acceptable carrier, a pharmaceutically acceptable diluent and/or a pharmaceutically acceptable excipient. In a further aspect, the kit is used for the manufacture of a general anesthetic.

The present invention also provides a selective κ-opioid receptor agonist for use in a method of inducing or maintaining a state of general anesthesia in a subject or animal, wherein the selective κ-opioid receptor agonist is co-administered with a α₂-adrenergic receptor agonist and/or a positive GABA_{A} receptor effector. Further, a α₂-adrenergic receptor agonist and/or a positive GABA_{A} receptor effector for use in a method of inducing or maintaining a state of general anesthesia in a subject or animal, wherein the α₂-adrenergic receptor agonist and/or the positive GABA_{A} receptor effector is co-administered with a selective κ-opioid receptor agonist is also provided by the present invention.

### DETAILED DESCRIPTION OF THE INVENTION

Definitions

The term “general anesthesia” refers to a state wherein a subject or animal exhibits (1) a loss of consciousness, (2) deep analgesia (patients cannot be aroused, even by painful stimulation), and (3) a suppression of voluntary movements and reflexes. The terms “sedation” and “analgesia” are not considered to be the same as the term “general anesthesia” because they do not fulfil all of the criteria that have been mentioned.

The term “general anesthetic” refers to a pharmaceutical composition which is able to induce and/or maintain general anesthesia in a subject or animal.

The terms “individual”, “patient” or “subject” are used interchangeably in the present application to designate a human being and are not meant to be limiting in any way. The “individual”, “patient” or “subject” can be of any age, sex and physical condition. The term “animal”, as used in the present application, refers to any multicellular eukaryotic heterotroph which is not a human. In a preferred embodiment, the animal is selected from a group consisting of cats, dogs, pigs, ferrets, rabbits, gerbils, hamsters, guinea pigs, horses, rats, mice, cows, sheep, goats, alpacas, camels, donkeys, llamas, yaks, giraffes, elephants, meerkats, lemurs, lions, tigers, kangaroos, koalas, bats, monkeys, chimpanzees, gorillas, bears, dugongs, manatees, seals and rhinoceroses.

The term “therapeutically effective amount” refers to an amount of combination product which is able to maintain and/or induce general anesthesia.

The term “combination product” can refer to (i) a product comprised of two or more regulated components that are physically, chemically, or otherwise combined or mixed and produced as a single entity; (ii) two or more separate products packaged together in a single package or as a unit and comprised of drug and device products, device and biological products, or biological and drug products; (iii) a drug, device, or biological product packaged separately that according to its investigational plan or proposed labeling is intended for use only with an approved individually specified drug, device, or biological product where both are required to achieve the intended use, indication, or effect and where upon approval of the proposed product the labeling of the approved product would need to be changed, e.g., to reflect a change in intended use, dosage form, strength, route of administration, or significant change in dose; or (iv) any investigational drug, device, or biological product packaged separately that according to its proposed labeling is for use only with another individually specified investigational drug, device, or biological product where both are required to achieve the intended use, indication, or effect.

As used herein, “pharmaceutically acceptable carrier” or “pharmaceutically acceptable diluent” means any and all solvents, dispersion media, coatings, antibacterial and antifungal agents, isotonic and absorption delaying agents, compatible with pharmaceutical administration. The use of such media and agents for pharmaceutically active substances is well known in the art. Acceptable carriers, excipients, or stabilizers are nontoxic to recipients at the dosages and concentrations employed and, without limiting the scope of the present invention, include: additional buffering agents; preservatives;

co-solvents; antioxidants, including ascorbic acid and methionine; chelating agents such as EDTA; metal complexes (e.g., Zn-protein complexes); biodegradable polymers, such as polyesters; salt-forming counterions, such as sodium, polyhydric sugar alcohols; amino acids, such as alanine, glycine, glutamine, asparagine, histidine, arginine, lysine, ornithine, leucine, 2-phenylalanine, glutamic acid, and threonine; organic sugars or sugar alcohols, such as lactitol, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, [alpha]-monothioglycerol, and sodium thio sulfate; low molecular weight proteins, such as human serum albumin, bovine serum albumin, gelatin, or other immunoglobulins; and hydrophilic polymers, such as polyvinylpyrrolidone. Other pharmaceutically acceptable carriers, excipients, or stabilizers, such as those described in Remington: The Science and Practice of Pharmacy 22^{nd} edition, Pharmaceutical press (2012), ISBN-13: 9780857110626 may also be included.

The term “receptor” refers to a protein molecule present on the membrane or in the interior of the cell that receives chemical signals (i.e., interacts with endogenous and/or exogenous molecules), leading to: a) the blockade of the said protein molecule (e.g. as caused by receptor antagonists); or b) a cellular response upon binding to the chemical signals (e.g. as caused by receptor agonists, partial agonists, inverse agonists and allosteric modulators).

The α₂-adrenergic receptor is a G protein-coupled receptor (GPCR). Its primary endogenous ligands are norepinephrine and epinephrine. There are three highly homologous subtypes including the α_{2A}- (e.g., UniProtKB—P08913), α_{2B}- (e.g., UniProtKB—P18089) and α_{2C}-adrenergic receptor (e.g., UniProtKB—P18825). The term “α₂-adrenergic receptor” may refer to any one or all of the subtypes. The term may also refer to a homologue in another species which has the same function as the α₂-adrenergic receptor in humans.

The GABA type A receptor (GABA_{A}) is an ionotropic receptor and ligand-gated ion channel. Its primary endogenous ligand is γ-aminobutyric acid (GABA), the major inhibitory neurotransmitter in the central nervous system. The GABA_{A} receptor is found in humans and the receptor has been sequenced and characterized. The GABA_{A} receptor comprises 8 known subunits (α, β, γ, δ, ε, θ, π and ρ), each presenting one or more isoforms. Data on each isoform have been deposited in the UniProtKB database under the following independent accession numbers: P14867 (α1), P47869 (α2), P34903 α3), P48169 (α4), P31644 (α5), Q16445 (α6), P18505 (β1), P47870 (β2), P28472 (β3), Q8N1C3 (γ1), P18507 (γ2), Q99928 (γ3), O14764 (δ), P78334 (ε), Q9UN88 (θ), O00591 (π), P24046 (ρ1) and P28476 (ρ2). The term “GABA_{A} receptor” may also refer to a homologue in another species which has the same function as the GABA_{A} receptor in humans.

The κ-opioid receptor (KOR) is a G protein-coupled receptor (GPCR). Its primary endogenous ligands are the opioid peptides known collectively as dynorphins. The KOR is found in humans and the receptor has been sequenced, characterized and the data have been deposited in the UniProtKB database under the accession number P41145. The term “KOR” may also refer to a homologue in another species which has the same function as the KOR in humans.

The term “receptor antagonist” as used in the present application refers to a type of receptor ligand and/or drug that blocks or dampens agonist- or partial agonist-mediated responses rather than provoking a biological response itself upon binding to a receptor. The term “receptor agonist” refers to a type of receptor ligand and/or drug that activates the receptor to produce a full (full agonist) or partial (partial agonist) biological response. As used in the present application, the term “receptor antagonist” may also refer to a type of receptor ligand and/or drug that activates the receptor to produce a biological response that is opposed to that produced by a full or partial agonist. Although these compounds are technically known as “inverse agonists”, here we use the term “receptor antagonist” to encompass both antagonists and inverse agonists. The reason being that some reports in the scientific literature initially labeled a given compound as an “antagonist”, while subsequent more detailed studies have found the same compound to display inverse agonist activity. Both antagonists and inverse agonists effectively counteract the effects of agonists (full or partial).

The terms “α₂-adrenergic receptor agonists” and “α₂-adrenergic agonists” refer to compounds that act predominantly on pre-synaptic receptors leading to reduced neuronal firing of adrenergic neurons (via auto-receptors) and non-adrenergic neurons (via hetero-receptors). Non-limiting examples of “α₂-adrenergic agonists” include: Medetomidine (CAS No. 86347-14-0), Dexmedetomidine (CAS No. 113775-47-6), Romifidine (CAS No. 65896-16-4), Detomidine (CAS No. 76631-46-4), Xylazine (CAS No. 7361-61-7), Clonidine (CAS No. 4205-90-7), Agmatine (CAS No. 306-60-5), Lofexidine (CAS No. 31036-80-3), Tizanidine (CAS No. 51322-75-9), Guanfacine (CAS No. 29110-47-2), Guanabenz (CAS No. 5051-62-7) and Mivazerol (CAS No. 125472-02-8).

The term “positive GABA_{A} receptor effector” refers to compounds that lead to neuron hyperpolarization and reduced neuronal firing through increased influx of chlorine ions into the cell. Positive GABA_{A} receptor effectors include positive allosteric modulators, agonists and partial agonists of the GABA_{A} receptor. Non-limiting examples of “positive GABA_{A} receptor effectors” include: Diazepam (CAS No. 439-14-5), Midazolam (CAS No. 59467-70-8), Lorazepam (CAS No. 846-49-1), Zolazepam (CAS No. 31352-82-6), Etomidate (CAS No.33125-97-2), Adinazolam (CAS No. 37115-32-5), Bentazepam (CAS No. 29462-18-8), Bromazepam (CAS No. 1812-30-2), Brotizolam (CAS No. 57801-81-7), Camazepam (CAS No. 36104-80-0), Chlorazepam (CAS No. 57109-90-7), Chlordiazepoxide (CAS No. 58-25-3), Cinolazepam (CAS No. 75696-02-5), Clobazam (CAS No. 22316-47-8), Clonazepam (CAS No. 1622-61-3), Clotiazepam (CAS No. 33671-46-4), Cloxazolam (CAS No. 24166-13-0), Estazolam (CAS No. 29975-16-4), Alprazolam (CAS No. 28981-97-7), Ethyl loflazepate (CAS No. 29177-84-2), Etizolam (CAS No. 40054-69-1), Fludiazepam (CAS No. 3900-31-0), Flunitrazepam (CAS No. 1622-62-4), Flurazepam (CAS No. 17617-23-1), Halazepam (CAS

No. 23092-17-3), Ketazolam (CAS No. 27223-35-4), Loprazolam (CAS No. 61197-73-7), Lormetazepam (CAS No. 848-75-9), Medazepam (CAS No. 2898-12-6), Nitrazepam (CAS No. 146-22-5), Nordiazepam (CAS No. 1088-11-5), Oxazepam (CAS No. 604-75-1), Pinazepam (CAS No. 52463-83-9), Prazepam (CAS No. 2955-38-6), Quazepam (CAS No. 36735-22-5), Temazepam (CAS No. 846-50-4), Tofisopam (CAS No. 22345-47-7), Triazolam (CAS No. 28911-01-5), Flutazolam (CAS No. 27060-91-9), Flutoprazepam (CAS No. 25967-29-7), Nimetazepam (CAS No. 2011-67-8), Mexazolam (CAS No. 31868-18-5), Haloxazolam (CAS No. 59128-97-1), Desflurane (CAS No. 57041-67-5), Enflurane (CAS No. 13838-16-9), Halothane (CAS No. 151-67-7), Isoflurane (CAS No. 26675-46-7), Methoxyflurane (CAS No. 76-38-0), Nitrous oxide (CAS No. 10024-97-2), Sevoflurane (CAS No. 28523-86-6), Thiopental (CAS No. 76-75-5), Thiopental sodium salt (CAS No. 71-73-8), Thiamylal (CAS No. 77-27-0), Pentobarbital (CAS No. 76-74-4), Secobarbital (CAS No. 76-73-3), Barbital (CAS No. 57-44-3), Methohexital (CAS No. 151-83-7), Chloral (CAS No. 75-87-6), Zaleplon (CAS No. 151319-34-5), Zolpidem (CAS No. 82626-48-0), Zopiclone (CAS No. 43200-80-2), Eszopiclone (CAS No. 138729-47-2), Desmetilzopiclone (CAS No. 59878-63-6), Indiplon (CAS No. 325715-02-4), Chloral hydrate (CAS No. 75-87-6), Triclofos (CAS No. 306-52-5), and Triclofos sodium salt (CAS No. 7246-20-0).

The term “selective κ-opioid receptor agonist” refers to an agonist that preferentially binds to the KOR over the μ-opioid receptor and/or δ-opioid receptor. The term excludes those compounds pertaining to the agonist-antagonist family of opioids. This drug class exhibits agonist activity at the KOR and antagonist activity at the MOR and/or DOR. Examples include pentazocine, butorphanol and nalbuphine. In a preferred embodiment, the selective KOR agonist shows at least five-fold greater affinity for the KOR than the MOR. This threshold has yielded adequate results to identify target-selective ligands of specific receptor subtypes (Kurczab et al., 2016). In a preferred embodiment, the selective KOR agonist shows at least 5-, 10-, 15- or 20-fold greater affinity for the KOR than the MOR and/or DOR.

Combination Product

In a first aspect, the present application provides a combination product comprising (i) one or more selective κ-opioid receptor agonists and (ii) one or more α₂-adrenergic receptor agonists and/or one or more positive GABA_{A} receptor effectors.

In a preferred embodiment, the selective κ-opioid receptor agonist is a terpene or terpenoid compound. Preferably, the selective κ-opioid receptor agonist is a diterpene or diterpenoid compound. Diterpenes comprise two terpene units or four isoprene units. Diterpenes are formally defined as hydrocarbons and therefore contain no heteroatoms whereas diterpenoids may be functionalized and may contain heteroatoms. More preferably, the selective κ-opioid receptor agonist is a clerodane diterpene or clerodane diterpenoid compound.

Non-limiting examples of clerodane diterpene or clerodane diterpenoid are disclosed in Table 2. Any clerodane diterpene or clerodane diterpenoid may be used as long as it selectively binds to KOR. Methods to determine whether a compound selectively binds to KOR instead of MOR or DOR are known in the art. For example, protocols are available at the PDSP (Psychoactive Drug Screening Program)—NIMH (National Institute of Mental Health) website (https://pdspdb.unc.edu/pdspWeb/). The website provides an assay protocol book (Roth, 2013. National Institute of Mental Health Psychoactive Drug Screening Program (NIMH PDSP) ASSAY PROTOCOL BOOK Version II. https://pdspdb.unc.eduipdspWeb/content/PDSP%20Protocols%20II%202013-03-28.pdf).

Methods of synthesizing clerodane diterpenes or clerodane diterpenoids are known in the art (see sources in Table 2). Further, the sorts of modifications which can alter the compound's ability to modulate KOR activity have been extensively studied and are known in the art (e.g., see FIG. 5 of Li et al., 2016, and Prisinzano and Rothman, 2008). Thus, it would not be an undue burden for a skilled person to identify or synthesize clerodane diterpenes or clerodane diterpenoids which are selective KOR agonists.

In a preferred embodiment, the selective κ-opioid receptor agonist is Salvinorin A or B, or analogue thereof. Non-limiting examples of analogues of Salvinorin A and B are provided in Table 2. In a preferred embodiment, the selective κ-opioid receptor agonist is a compound described by the following formula (I):

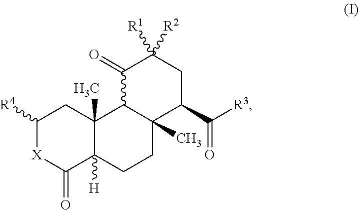

wherein R1, R2, R3 and R4 are selected, independently, from Table 1 and X is C or O, or R3 and R4 are selected, independently, from Table 1, X is C or O, and R1 and R2 form a 3-5 membered alkyl ring which may be substituted with 0 and comprises at least one heteroatom which is an O (see compound 90 for an example); or

the selective κ-opioid receptor agonist is a compound described by the following formula (II):

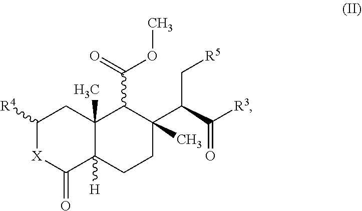

wherein:

R3 and R4 are selected, independently, from Table 1, X is C or O, and R5 is selected from the group consisting of C═O, CH₂OAc and CH(OMe)₂.

TABLE 1

| R1 | R2 | R3 | R4 |
|---|---|---|---|
| (1) *—H | (1) *—OH | 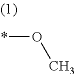 [SMILES: *OC] | 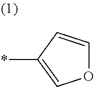 [SMILES: *c1ccoc1] |
| (2) *—CH₃ | 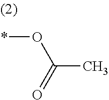 [SMILES: *OC(C)=O] | 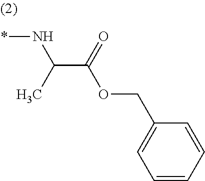 [SMILES: *NC(C)C(=O)OCc1ccccc1] | 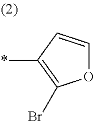 [SMILES: *c1ccoc1Br] |
| 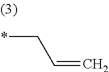 [SMILES: *CC=C] | 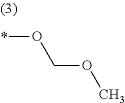 [SMILES: *OCOC] | (3) *—OH | 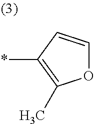 [SMILES: *c1ccoc1C] |
| | 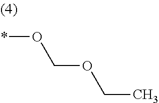 [SMILES: *OCOCC] | | 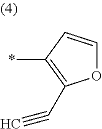 [SMILES: *c1ccoc1C#C] |
| | 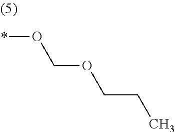 [SMILES: *OCOCCC] | | 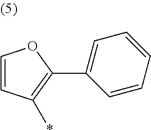 [SMILES: *c1ccoc1-c1ccccc1] |
| | 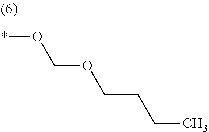 [SMILES: *OCOCCCC] | | 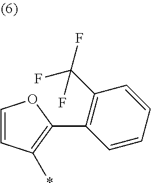 [SMILES: *c1ccoc1-c1ccccc1C(F)(F)F] |
| | 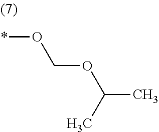 [SMILES: *OCOC(C)C] | | 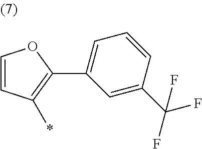 [SMILES: *c1ccoc1-c1cccc(C(F)(F)F)c1] |
| | 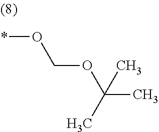 [SMILES: *OCOC(C)(C)C] | | 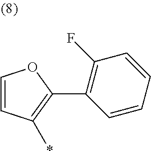 [SMILES: *c1ccoc1-c1ccccc1F] |
| | 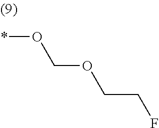 [SMILES: *OCOCCF] | | 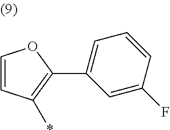 [SMILES: *c1ccoc1-c1cccc(F)c1] |
| | 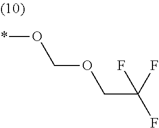 [SMILES: *OCOCC(F)(F)F] | | 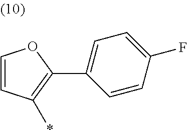 [SMILES: *c1ccoc1-c1ccc(F)cc1] |
| | 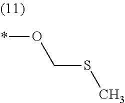 [SMILES: *OCSC] | | 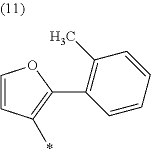 [SMILES: *c1ccoc1-c1ccccc1C] |
| | 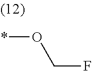 [SMILES: *OCF] | | 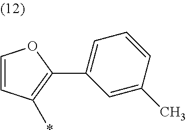 [SMILES: *c1ccoc1-c1cccc(C)c1] |
| | 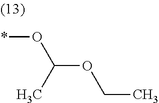 [SMILES: *OC(C)OCC] | | 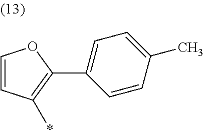 [SMILES: *c1ccoc1-c1ccc(C)cc1] |
| | 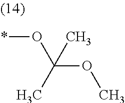 [SMILES: *OC(C)(C)OC] | | 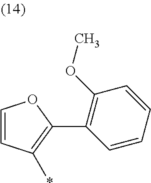 [SMILES: *c1ccoc1-c1ccccc1OC] |
| | 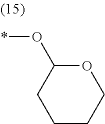 [SMILES: *OC1CCCCO1] | | 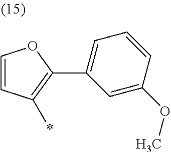 [SMILES: *c1ccoc1-c1cccc(OC)c1] |
| | 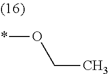 [SMILES: *OCC] | | 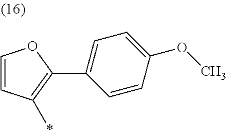 [SMILES: *c1ccoc1-c1ccc(OC)cc1] |
| | 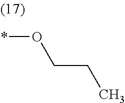 [SMILES: *OCCC] | | 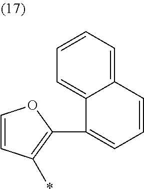 [SMILES: *c1ccoc1-c1cccc2ccccc12] |
| | 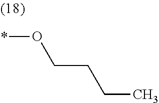 [SMILES: *OCCCC] | | 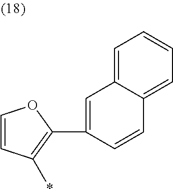 [SMILES: *c1ccoc1-c1ccc2ccccc2c1] |
| | 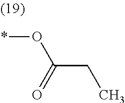 [SMILES: *OC(=O)CC] | | 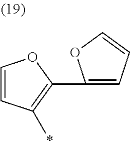 [SMILES: *c1ccoc1-c1ccco1] |
| | 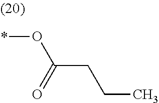 [SMILES: *OC(=O)CCC] | | 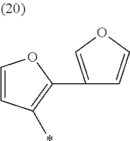 [SMILES: *c1ccoc1-c1ccoc1] |
| | 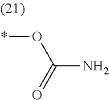 [SMILES: *OC(N)=O] | | 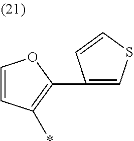 [SMILES: *c1ccoc1-c1ccsc1] |
| | 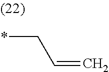 [SMILES: *CC=C] | | 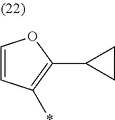 [SMILES: *c1ccoc1C1CC1] |
| | 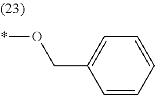 [SMILES: *OCc1ccccc1] | | 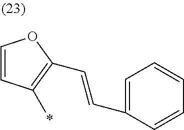 [SMILES: *c1ccoc1/C=C/c1ccccc1] |
| | 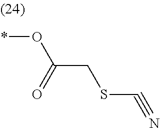 [SMILES: *OC(=O)CSC#N] | | 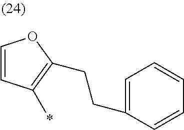 [SMILES: *c1ccoc1CCc1ccccc1] |
| | 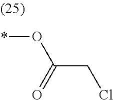 [SMILES: *OC(=O)CCl] | | 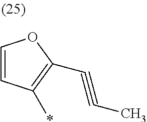 [SMILES: *c1ccoc1C#CC] |
| | 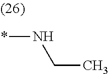 [SMILES: *NCC] | | 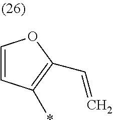 [SMILES: *c1ccoc1C=C] |
| | 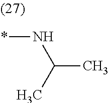 [SMILES: *NC(C)C] | | 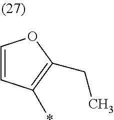 [SMILES: *c1ccoc1CC] |
| | 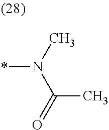 [SMILES: *N(C)C(C)=O] | | 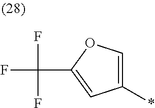 [SMILES: *c1coc(C(F)(F)F)c1] |
| | 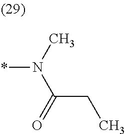 [SMILES: *N(C)C(=O)CC] | | 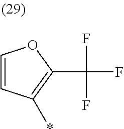 [SMILES: *c1ccoc1C(F)(F)F] |
| | 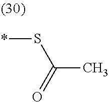 [SMILES: *SC(C)=O] | | 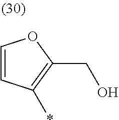 [SMILES: *c1ccoc1CO] |
| | (31) *—SH | | 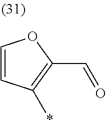 [SMILES: *c1ccoc1C=O] |
| | 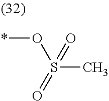 [SMILES: *OS(C)(=O)=O] | | 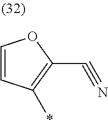 [SMILES: *c1ccoc1C#N] |
| | 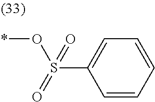 [SMILES: *OS(=O)(=O)c1ccccc1] | | 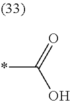 [SMILES: *C(=O)O] |
| | 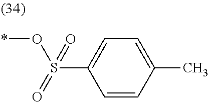 [SMILES: *OS(=O)(=O)c1ccc(C)cc1] | | 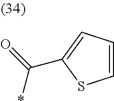 [SMILES: *C(=O)c1cccs1] |
| | 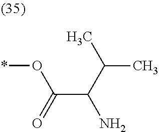 [SMILES: *OC(=O)C(N)C(C)C] | | 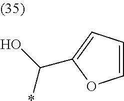 [SMILES: *C(O)c1ccco1] |
| | 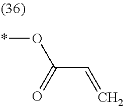 [SMILES: *OC(=O)C=C] | | 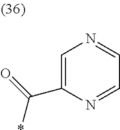 [SMILES: *C(=O)c1cnccn1] |
| | 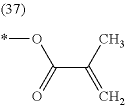 [SMILES: *OC(=O)C(=C)C] | | 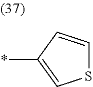 [SMILES: *c1ccsc1] |
| | 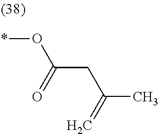 [SMILES: *OC(=O)CC(=C)C] | | 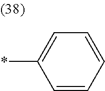 [SMILES: *c1ccccc1] |
| | 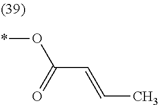 [SMILES: *OC(=O)/C=C/C] | | |
| | 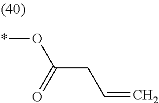 [SMILES: *OC(=O)CC=C] | | |
| | 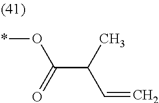 [SMILES: *OC(=O)C(C)C=C] | | |
| | 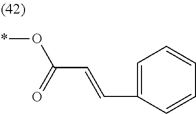 [SMILES: *OC(=O)/C=C/c1ccccc1] | | |
| | 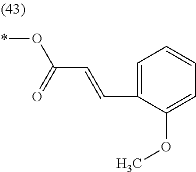 [SMILES: *OC(=O)/C=C/c1ccccc1OC] | | |

In a preferred embodiment, the selective κ-opioid receptor agonist is selected from Table 2.

TABLE 2

| | | PubChem | | |
|---|---|---|---|---|
| No. | Name | CID | Structure | Source |
| 1 | Salvinorin A | 128563 | 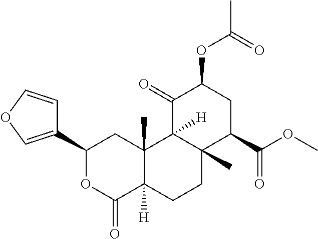 | Commercial: Sigma-Aldrich Cat. No. S8071 Extraction: Ortega et al., 1982; Valdes et al., 1984; Munro and Rizzacasa, 2003 Synthesis: Nozawa et al., 2008 |
| 2 | Salvinorin B | 11440685 | 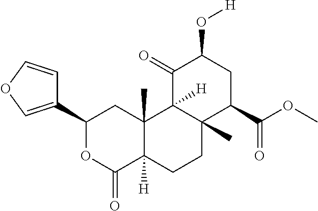 | Commercial: Sigma-Aldrich Cat. No. 75250 Synthesis: Tidgewell et al., 2004 |
| 3 | Salvinorin B methoxymethyl ether | 44456192 | 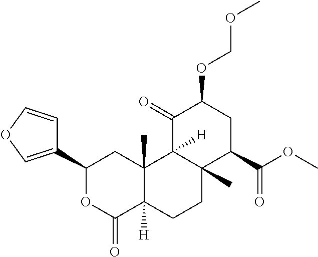 | Synthesis: Munro et al., 2008 |
| 4 | Salvinorin B ethoxymethyl ether | 24873526 | 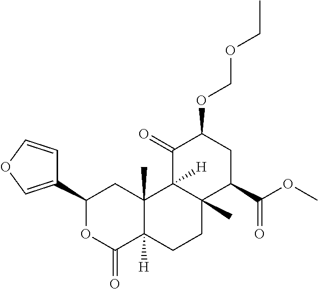 | Synthesis: Munro et al., 2008 |
| 5 | Salvinorin B propoxymethyl ether | 44456420 | 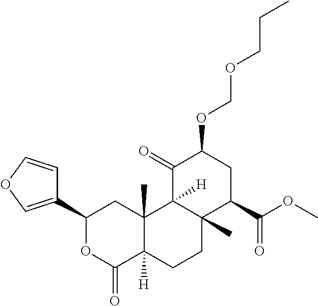 | Synthesis: Munro et al., 2008 |
| 6 | Salvinorin B butoxymethyl ether | 44456421 | 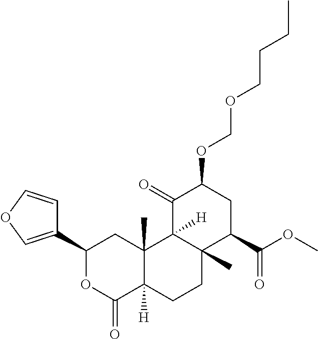 | Synthesis: Munro et al., 2008 |
| 7 | Salvinorin B isopropoxymethyl ether | 44456377 | 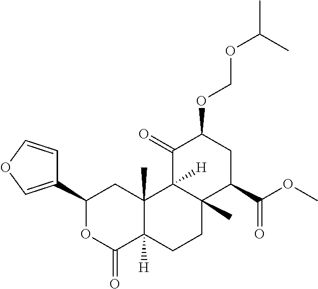 | Synthesis: Munro et al., 2008 |
| 8 | Salvinorin B tert-butoxymethyl ether | 44456378 | 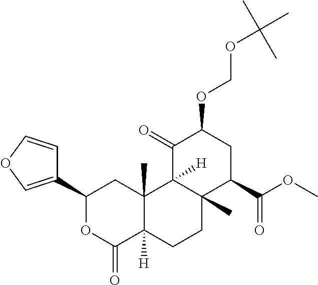 | Synthesis: Munro et al., 2008 |
| 9 | Salvinorin B 2- fluoroethoxymethyl ether | 44456375 | 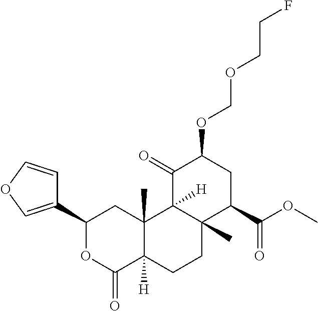 | Synthesis: Munro et al., 2008 |
| 10 | Salvinorin B 2,2,2- trifluoroethoxymethyl ether | 44456346 | 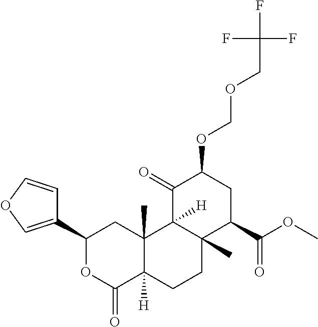 | Synthesis: Munro et al., 2008 |
| 11 | Salvinorin B methylthiomethyl ether | 44456307 | 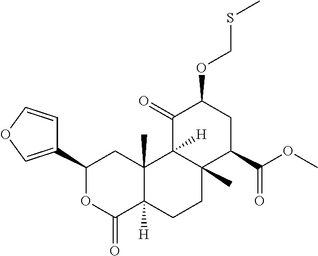 | Synthesis: Munro et al., 2008 |
| 12 | Salvinorin B fluoromethyl ether | 44456305 | 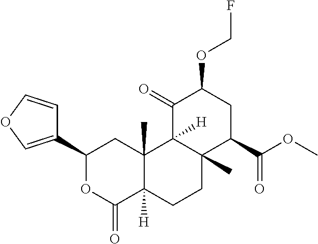 | Synthesis: Munro et al., 2008 |
| 13 | Salvinorin B 1-ethoxyethyl ether | 45266000 | 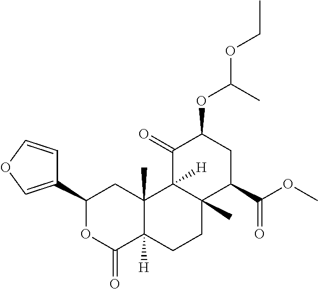 | Synthesis: Munro et al., 2008 |
| 14 | Salvinorin B 2-methoxy-2- propyl ether | 44456105 | 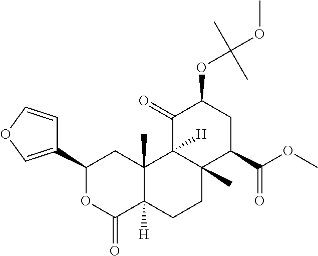 | Synthesis: Munro et al., 2008 |
| 15 | Salvinorin B tetrahydropyran- 2-yl ether | 44456106 | 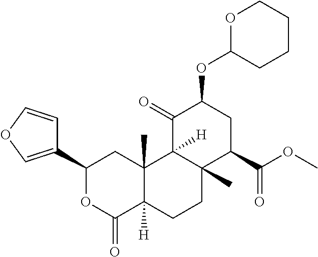 | Synthesis: Munro et al., 2008 |
| 16 | 2-ethoxy-Salvinorin B | 44402661 | 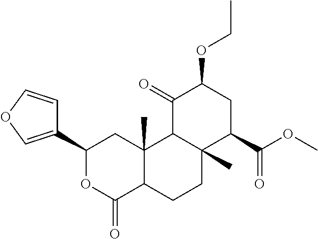 [SMILES: CCO[C@H]1C[C@@H](C(=O)OC)[C@]2(C)CCC3C(=O)O[C@@H](c4ccoc4)C[C@]3(C)C2C1=O] | Synthesis: Béguin et al., 2005; WO 2005/089745 A1 |
| 17 | 2-propoxy-Salvinorin B | 44402551 | 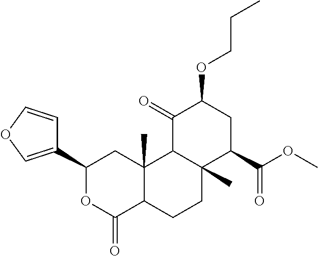 [SMILES: CCCO[C@H]1C[C@@H](C(=O)OC)[C@]2(C)CCC3C(=O)O[C@@H](c4ccoc4)C[C@]3(C)C2C1=O] | Synthesis: Béguin et al., 2005; WO 2005/089745 A1 |
| 18 | 2-n-butoxy-Salvinorin B | 44402440 | 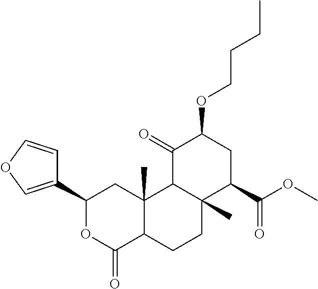 [SMILES: CCCCO[C@H]1C[C@@H](C(=O)OC)[C@]2(C)CCC3C(=O)O[C@@H](c4ccoc4)C[C@]3(C)C2C1=O] | Synthesis: Béguin et al., 2005; WO 2005/089745 A1 |
| 19 | 2-propionyl Salvinorin B | 44402421 | 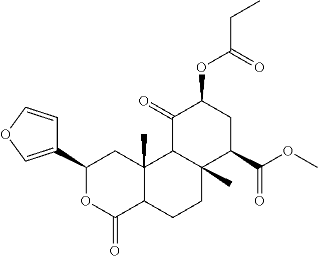 [SMILES: CCC(=O)O[C@H]1C[C@@H](C(=O)OC)[C@]2(C)CCC3C(=O)O[C@@H](c4ccoc4)C[C@]3(C)C2C1=O] | Synthesis: Béguin et al., 2005; Harding et al., 2005; WO 2005/089745 A1 |
| 20 | 2-butanoyl Salvinorin B | 44402663 | 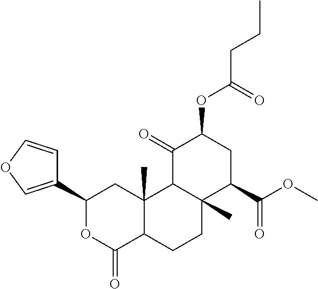 [SMILES: CCCC(=O)O[C@H]1C[C@@H](C(=O)OC)[C@]2(C)CCC3C(=O)O[C@@H](c4ccoc4)C[C@]3(C)C2C1=O] | Synthesis: Béguin et al., 2005; WO 2005/089745 A1 |
| 21 | 2-(O-formamide)-Salvinorin B | 11384968 | 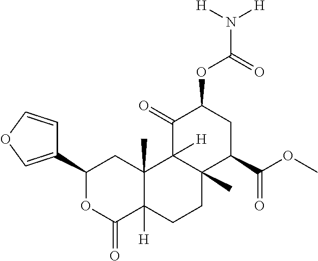 | Synthesis: Béguin et al., 2005; WO 2005/089745 A1 |
| 22 | 2-allyloxy Salvinorin B | 118718688 | 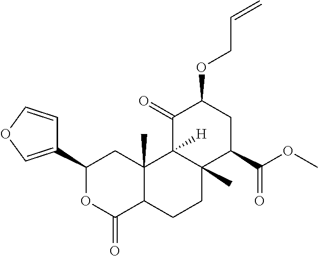 | Synthesis: Béguin et al., 2005; WO 2005/089745 A1 |
| 23 | 2-benzyloxy Salvinorin B | 118718691 | 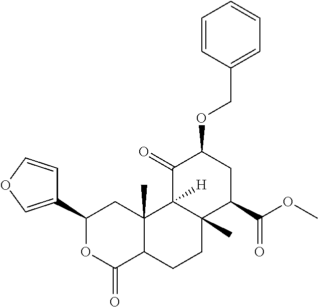 | Synthesis: Béguin et al., 2005; WO 2005/089745 A1 |
| 24 | RB-64 | 73347341 | 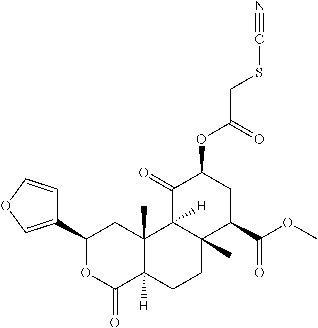 | Synthesis: Yan et al., 2009; Polepally et al., 2013 |
| 25 | RB-48 | 101482513 | 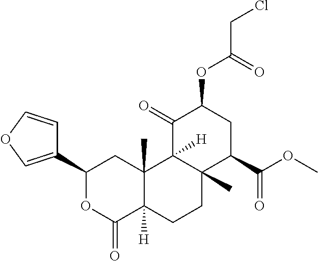 | Synthesis: Yan et al., 2009 |
| 26 | 2S-(N-ethylamino)-Salvinorin B | 44415874 | 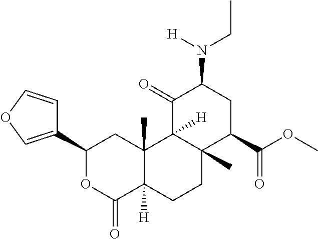 | Synthesis: Béguin et al., 2006; WO 2005/089745 A1 |
| 27 | 2S-(N-isopropylamino)- Salvinorin B | 44415888 | 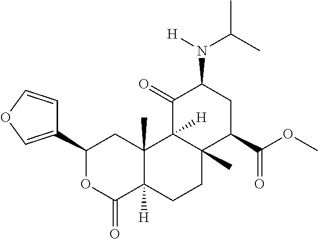 | Synthesis: Béguin et al., 2006; WO 2005/089745 A1 |
| 28 | 9S-[acetyl(methyl)amino]- Salvinorin B | 44416030 | 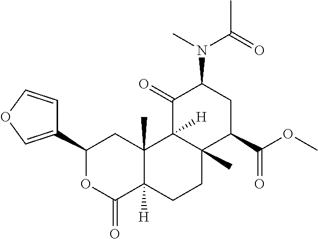 | Synthesis: Béguin et al., 2006; WO 2005/089745 A1 |
| 29 | 2-epi-(N-ethylamino)- Salvinorin B | 44415932 | 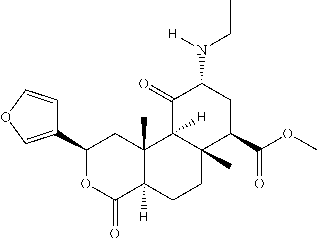 | Synthesis: Béguin et al., 2005; Béguin et al., 2006; WO 2005/089745 A1 |
| 30 | | 44415946 | 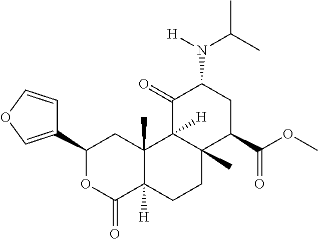 | Synthesis: Béguin et al., 2006; WO 2005/089745 A1 |
| 31 | | 44416145 | 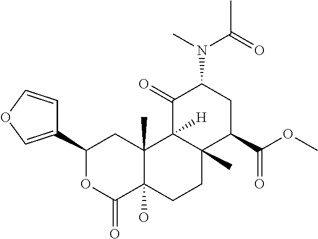 | Synthesis: Béguin et al., 2006; WO 2005/089745 A1 |
| 32 | | 44415854 | 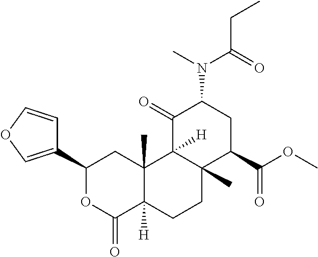 | Synthesis: Béguin et al., 2006; WO 2005/089745 A1 |
| 33 | | 44403387 | 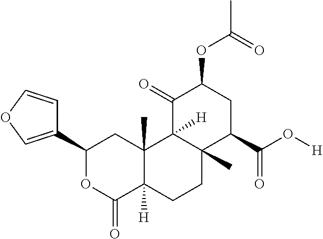 | Synthesis: WO 2005/089745 A1 |
| 34 | | 44403459 | 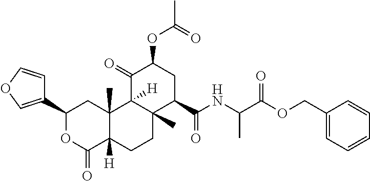 | Synthesis: WO 2005/089745 A1 |
| 35 | | 44403458 | 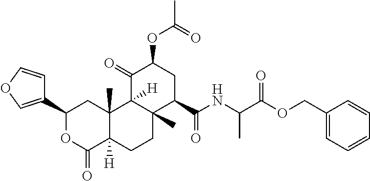 | Synthesis: WO 2005/089745 A1 |
| 36 | | 11534360 | 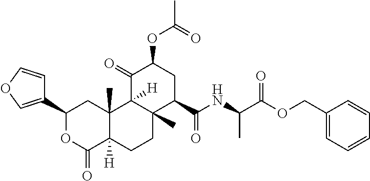 | Synthesis: WO 2005/089745 A1 |
| 37 | Episalvinorin B | 11395172 | 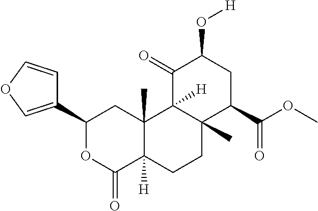 | Synthesis: WO 2005/089745 A1 |
| 38 | 2-methoxymethyl-epi- salvinorin B | | 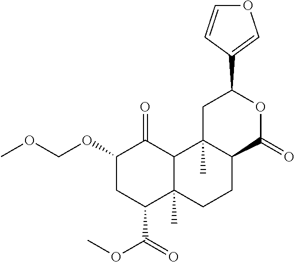 | Synthesis: WO 2005/089745 A1 |
| 39 | Salvinorinyl-2-thioacetate | 11510652 | 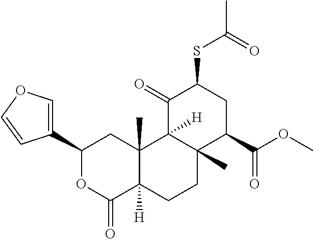 | Synthesis: Stewart et al., 2006; WO 2006/012643 A2 |
| 40 | 2-thiosalvinorin B | 17747979 | 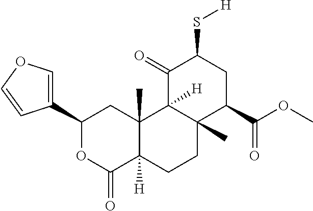 | Synthesis: Bikbulatov et al., 2007; WO 2006/012643 A2 |
| 41 | 16-bromo-Salvinorin A | 11526334 | 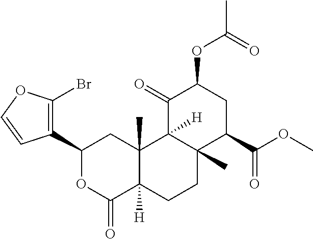 | Synthesis: Riley et al., 2013; Riley et al., 2014; US 2012/0010219 A1 |
| 42 | 16-methyl-Salvinorin A | 73212901 | 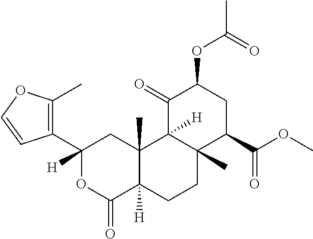 | Synthesis: Riley et al., 2013; Riley et al., 2014 |
| 43 | 16-ethynyl-Salvinorin A | 101910788 | 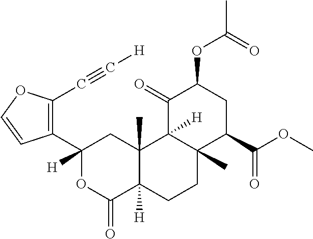 | Synthesis: Riley et al., 2013; Riley et al., 2014 |
| 44 | | 73212818 | 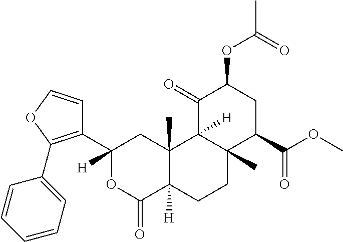 | Synthesis: Riley et al., 2013; Riley et al., 2014 |
| 45 | | 118723390 | 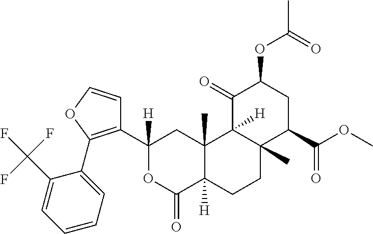 | Synthesis: Riley et al., 2013; Riley et al., 2014 |
| 46 | | 118723391 | 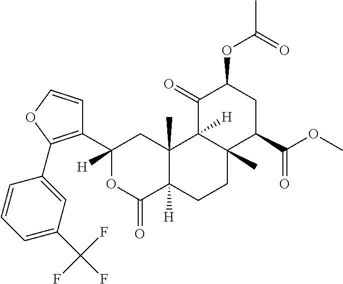 | Synthesis: Riley et al., 2013; Riley et al., 2014 |
| 47 | | 118723393 | 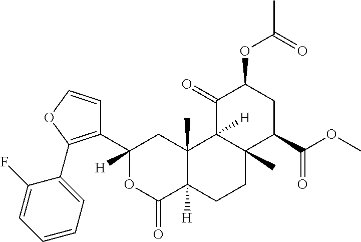 | Synthesis: Riley et al., 2013; Riley et al., 2014 |
| 48 | | 118723394 | 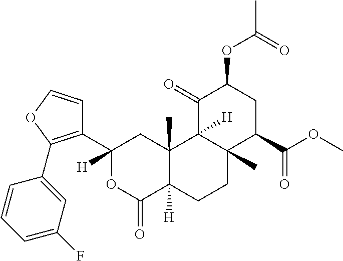 | Synthesis: Riley et al., 2013; Riley et al., 2014 |
| 49 | | 118723395 | 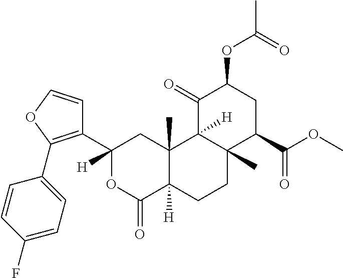 | Synthesis: Riley et al., 2013; Riley et al., 2014 |
| 50 | | 118723399 | 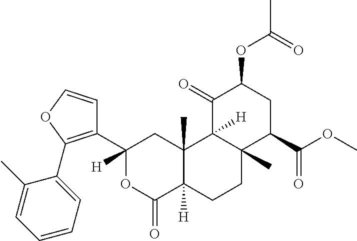 | Synthesis: Riley et al., 2013; Riley et al., 2014 |
| 51 | | 118723400 | 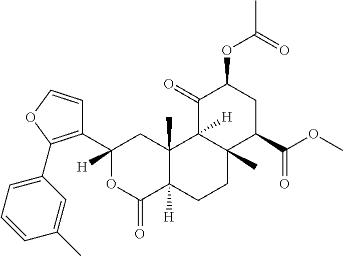 | Synthesis: Riley et al., 2013; Riley et al., 2014 |
| 52 | | 118723401 | 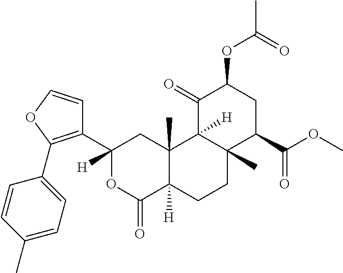 | Synthesis: Riley et al., 2013; Riley et al., 2014 |
| 53 | | 118723402 | 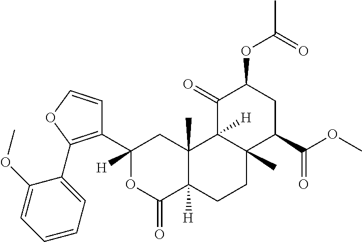 | Synthesis: Riley et al., 2013; Riley et al., 2014 |
| 54 | | 118723403 | 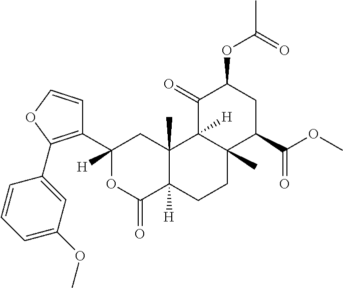 | Synthesis: Riley et al., 2013; Riley et al., 2014 |
| 55 | | 73212819 | 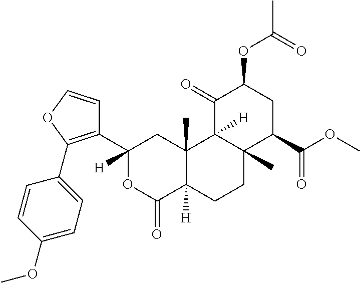 | Synthesis: Riley et al., 2013; Riley et al., 2014 |
| 56 | | 118723404 | 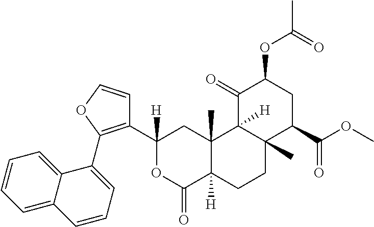 | Synthesis: Riley et al., 2013; Riley et al., 2014 |
| 57 | | 118723405 | 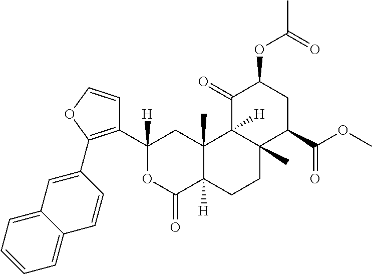 | Synthesis: Riley et al., 2013; Riley et al., 2014 |
| 58 | | 73212820 | 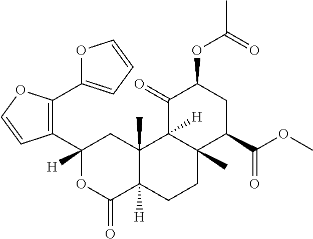 | Synthesis: Riley et al., 2013; Riley et al., 2014 |
| 59 | | 118723840 | 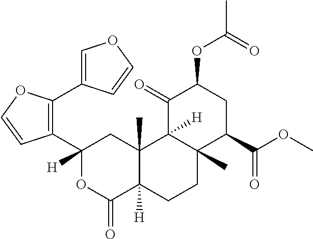 | Synthesis: Riley et al., 2013; Riley et al., 2014 |
| 60 | | 118723841 | 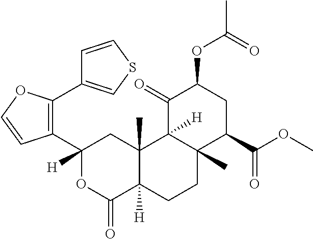 | Synthesis: Riley et al., 2013; Riley et al., 2014 |
| 61 | | 118723842 | 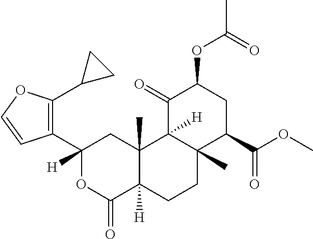 | Synthesis: Riley et al., 2013; Riley et al., 2014 |
| 62 | | 118723843 | 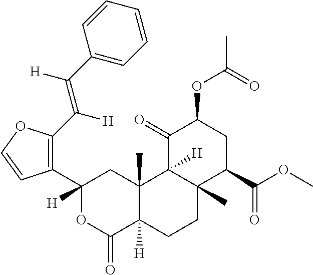 | Synthesis: Riley et al., 2013; Riley et al., 2014 |
| 63 | | 118723844 | 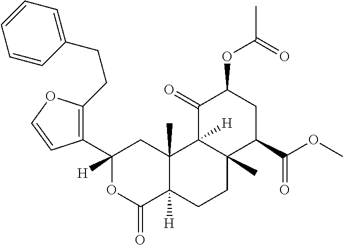 | Synthesis: Riley et al., 2013; Riley et al., 2014 |
| 64 | | 118723845 | 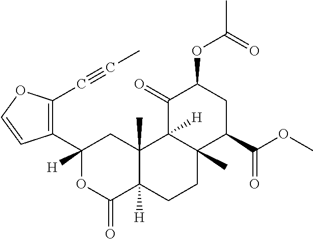 | Synthesis: Riley et al., 2013; Riley et al., 2014 |
| 65 | | 44581771 | 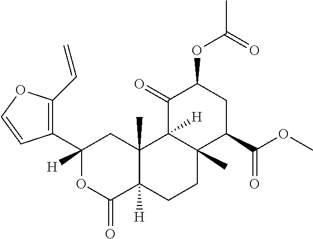 | Synthesis: Riley et al., 2013; Riley et al., 2014; WO 2010/075045 A1 |
| 66 | | 118723850 | 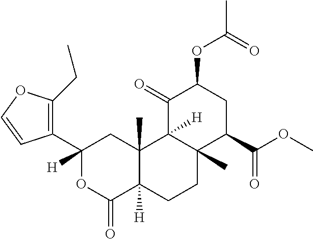 | Synthesis: Riley et al., 2013; Riley et al., 2014 |
| 67 | | 118723856 | 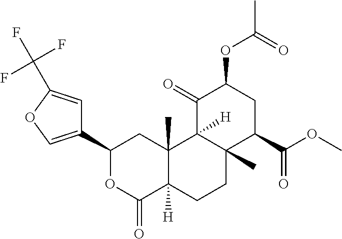 | Synthesis: Riley et al., 2013; Riley et al., 2014 |
| 68 | | 118723857 | 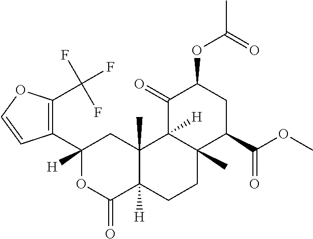 | Synthesis: Riley et al., 2013; Riley et al., 2014 |
| 69 | | 101886614 | 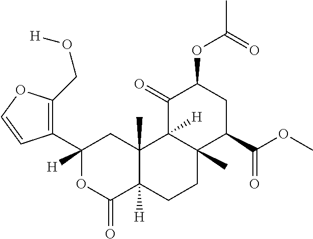 | Synthesis: Riley et al., 2013; Riley et al., 2014 |
| 70 | | 118723858 | 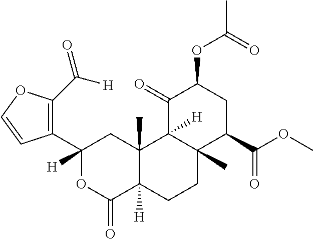 | Synthesis: Riley et al., 2013; Riley et al., 2014 |
| 71 | | 118723859 | 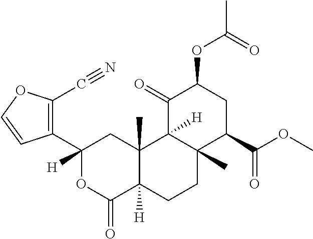 | Synthesis: Riley et al., 2013; Riley et al., 2014 |
| 72 | | 11509780 | 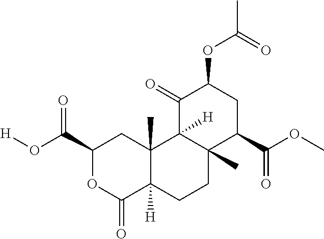 | Synthesis: Harding et al., 2006 |
| 73 | 12-epi-Salvinorin A | 25242485 | 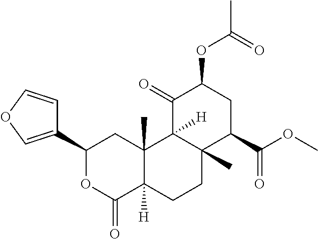 | Synthesis: WO 2010/075045 A1 |
| 74 | | 44581834 | 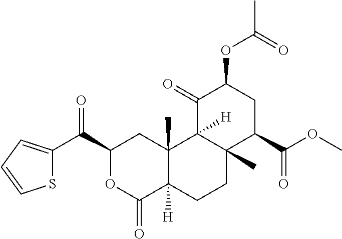 | Synthesis: WO 2010/075045 A1 |
| 75 | | 44581871 | 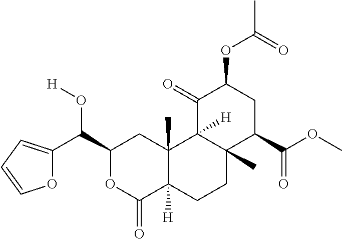 | Synthesis: WO 2010/075045 A1 |
| 76 | | 44581870 | 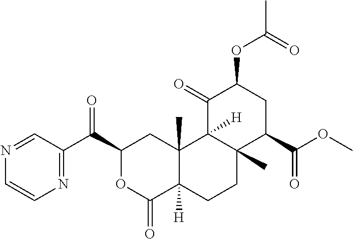 | Synthesis: WO 2010/075045 A1 |
| 77 | 12-epi-Salvinorin-B- methoxymethyl ether | 46831301 | 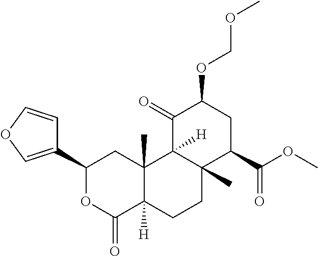 | Synthesis: Béguin et al., 2012 |
| 78 | 12-epi-Salvinorin-B- ethoxymethyl ether | 56951181 | 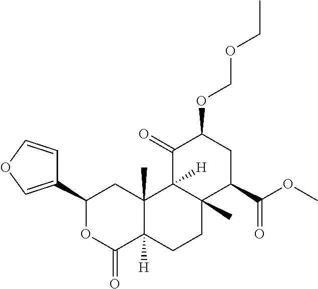 | Synthesis: Béguin et al., 2012 |
| 79 | 2-O-acryloylsalvinorin B | 118713122 | 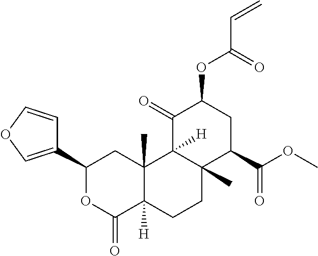 | Synthesis: Polepally et al., 2014 |
| 80 | 2-O-Methacryloylsalvinorin B | 11487946 | 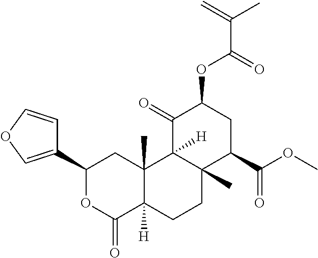 | Synthesis: Polepally et al., 2014 |
| 81 | 2-O-(3′-Methylbut-3′- enoyl)salvinorin B | 118713124 | 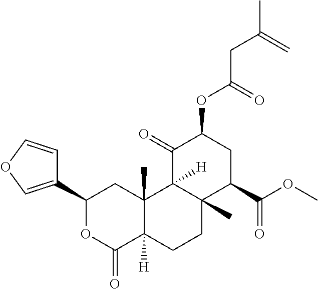 | Synthesis: Polepally et al., 2014 |
| 82 | 2-O-Crotonoylsalvinorin B | 118713125 | 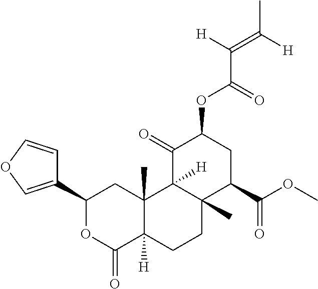 | Synthesis: Polepally et al., 2014 |
| 83 | 2-O-(3′-Butenoyl)salvinorin B | 118713126 | 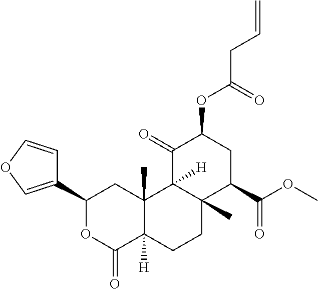 | Synthesis: Polepally et al., 2014 |
| 84 | 2-O-(2′-Methyl-3′- butenoyl)salvinorin B | 118713128 | 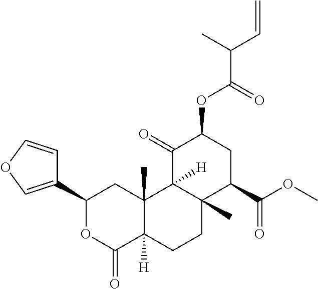 | Synthesis: Polepally et al., 2014 |
| 85 | 2-O-Cinnamoylsalvinorin B | 118713129 | 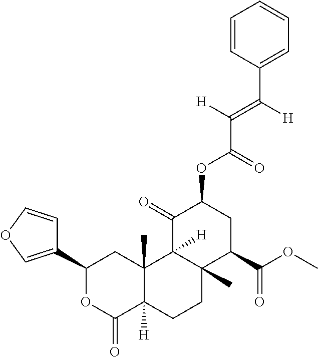 | Synthesis: Polepally et al., 2014 |
| 86 | 2-O-(2″- Methoxycinnamoyl)salvinorin B | 118713130 | 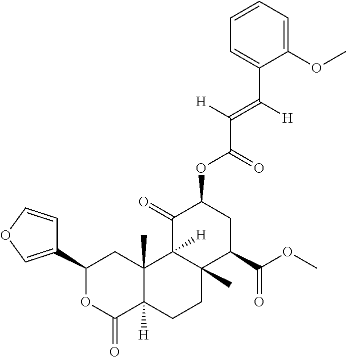 | Synthesis: Polepally et al., 2014 |
| 87 | | 122191992 | 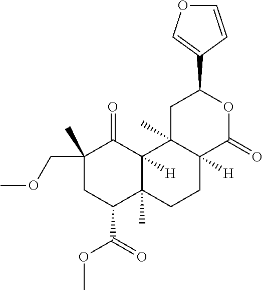 | Synthesis: Lee et al., 2015 |
| 88 | | 122191991 | 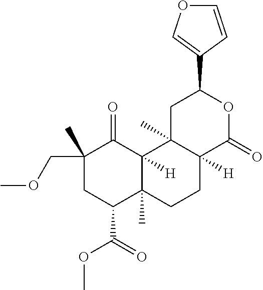 | Synthesis: Lee et al., 2015 |
| 89 | 2-allyl-2-methoxymethyl- Salvinorin-B ether | 122191993 | 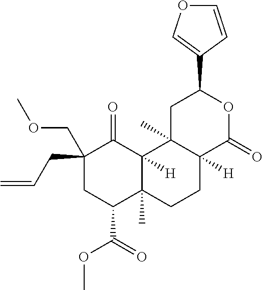 | Synthesis: Lee et al., 2015 |
| 90 | Methyl (2S,4aR,6aR,7R,9R,10aS,10bR)- 2-(Furan-3-yl)-6a,10b-dimethyl- 4,5′,10-trioxododecahydro- 2H,3′H,4H-spiro[benzo[f]- isochromene-9,2′-furan]-7- carboxylate | | 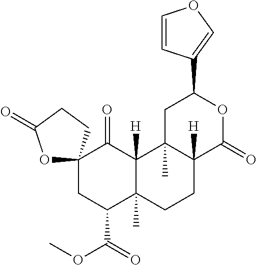 | Synthesis: Sherwood et al., 2017a |
| 91 | Methyl (3S,4aS,5S,6S,8aR)-3- (Furan-3-yl)-6-((R)-1-methoxy- 1,4-dioxobutan-2-yl)-4a,6- dimethyl-1-oxooctahydro-1H- isochromene-5-carboxylate | | 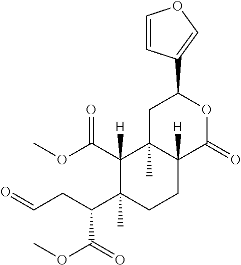 | Synthesis: Sherwood et al., 2017a |
| 92 | Methyl (3S,4aS,5S,6S,8aR)-6- ((R)-4-Acetoxy-1-methoxy-1- oxobutan-2-yl)-3-(furan-3-yl)- 4a,6-dimethyl-1-oxooctahydro- 1H-isochromene-5-carboxylate | | 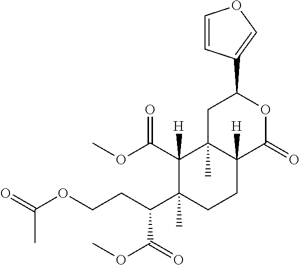 | Synthesis: Sherwood et al., 2017a |
| 93 | Methyl (3S,4aS,5S,6S,8aR)-3- (Furan-3-yl)-4a,6-dimethyl-1- oxo-6-((R)-1,4,4-trimethoxy-1- oxobutan-2-yl)octahydro-1H- isochromene-5-carboxylate | | 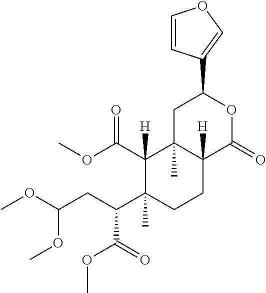 | Synthesis: Sherwood et al., 2017a |
| 94 | Methanesulfonyl-Salvinorin B | 11271318 | 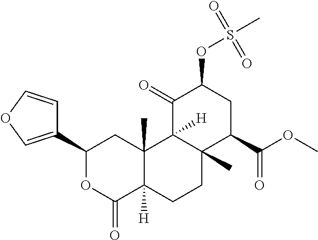 | Synthesis: Harding et al., 2005; WO 2006/031782 A2 |
| 95 | 8-epi-1-Salvinorin A | 21589297 | 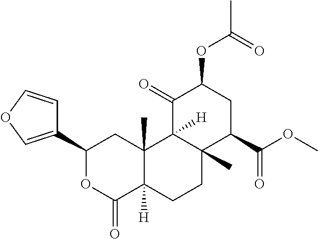 | Synthesis: Munro et al., 2005 |
| 96 | | 11591838 | 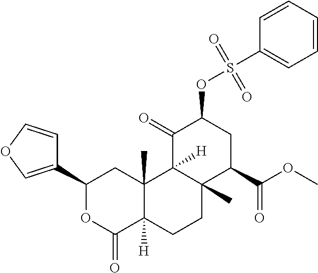 | Synthesis: Tidgewell et al., 2006 |
| 97 | | 11533999 | 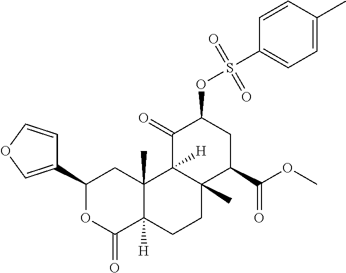 | Synthesis: Tidgewell et al., 2006 |
| 98 | | 50942591 | 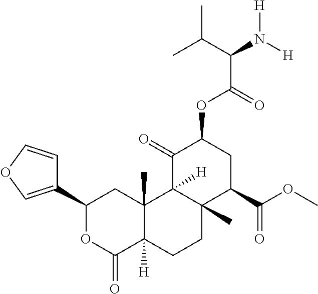 | Synthesis: Fichna et al., 2011 |
| 99 | (2S,6aS,7R,9S,10aS,10bR)-2- (furan-3-yl)-5-hydroxy-9- (methoxymethoxy)-7- (methoxymethyl)- 1,2,6a,7,8,9,10a,10b-octahydro- 4H-benzo[f]isochromene- 4,10(6H)-dione | | 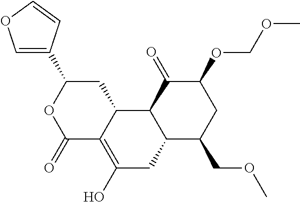 [SMILES: COCO[C@H]1C[C@@H](COC)[C@@H]2CC(O)=C3C(=O)O[C@H](c4ccoc4)C[C@@H]3[C@H]2C1=O] | Synthesis: Sherwood et al., 2017b |
| 100 | 20-nor-Salvinorin A | | 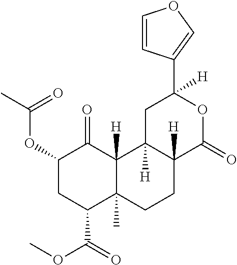 | Synthesis: Roach et al., 2017 |
| 101 | 12-epi-20-nor-Salvinorin A | | 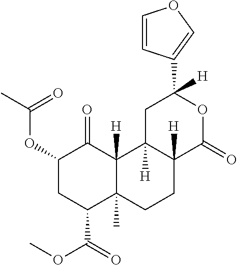 | Synthesis: Roach et al., 2017 |
| 102 | Named “13” in Roach et al., 2017 | | 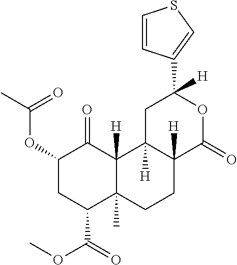 | Synthesis: Roach et al., 2017 |
| 103 | Named “14” in Roach et al., 2017 | | 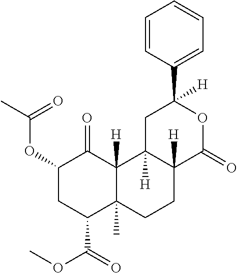 | Synthesis: Roach et al., 2017 |
| 104 | 12-epi-14 | | 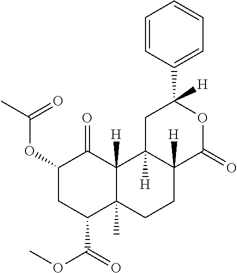 | Synthesis: Roach et al., 2017 |
| 105 | O6C-20-nor-salvinorin A | | 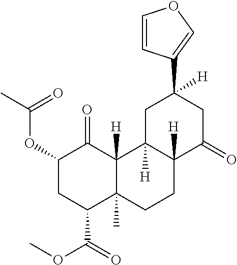 | Synthesis: Hirasawa et al., 2018 |

Preferably, the selective KOR agonist is selected from a group consisting of compounds No. 1, 3, 4, 24, 41-71, 77-81, 83, 87, 90, 94, 100, and 102-104 of Table 2. More preferably, the selective KOR agonist is selected from a group consisting of compounds No. 1, 3, 4, 41, 43, 61, 65, and 100 of Table 2.

In a preferred embodiment, the selective KOR agonist is Salvinorin A.

In a preferred embodiment, the α₂-adrenergic receptor agonist is selected from a group consisting of Medetomidine, Dexmedetomidine, Romifidine, Detomidine, Xylazine, Clonidine, Agmatine, Lofexidine, Tizanidine, Guanfacine, Guanabenz and Mivazerol. Preferably, the α₂-adrenergic receptor agonist is selected from a group consisting of Xylazine, Romifidine, Detomidine and Medetomidine. More preferably, the α₂-adrenergic receptor agonist is Medetomidine.

In a preferred embodiment, the positive GABA_{A} receptor effector is selected from a group consisting of Diazepam, Midazolam, Lorazepam, Zolazepam, Etomidate, Adinazolam, Bentazepam, Bromazepam, Brotizolam, Camazepam, Chlorazepam, Chlordiazepoxide, Cinolazepam, Clobazam, Clonazepam, Clotiazepam, Cloxazolam, Estazolam, Alprazolam, Ethyl loflazepate, Etizolam, Fludiazepam, Flunitrazepam, Flurazepam, Halazepam, Ketazolam, Loprazolam, Lormetazepam, Medazepam, Nitrazepam, Nordiazepam, Oxazepam, Pinazepam, Prazepam, Quazepam, Temazepam, Tofisopam, Triazolam, Flutazolam, Flutoprazepam, Nimetazepam, Mexazolam, Haloxazolam, Desflurane, Enflurane, Halothane, Isoflurane, Methoxyflurane, Nitrous oxide, Sevoflurane, Thiopental, Thiopental sodium salt, Thiamylal, Pentobarbital, Secobarbital, Barbital, Methohexital, Chloral, Zaleplon, Zolpidem, Zopiclone, Eszopiclone, Desmetilzopiclone, Indiplon, Chloral hydrate, Triclofos, and Triclofos sodium salt. Preferably, the positive GABA_{A} receptor effector is Diazepam.

In an alternative embodiment, the positive GABA_{A} receptor effector is a benzodiazepine or analogue thereof Preferably, the benzodiazepine or analogue thereof is selected from a group consisting of Diazepam, Midazolam, Lorazepam, Zolazepam, Adinazolam, Bentazepam, Bromazepam, Brotizolam, Camazepam, Chlorazepam, Chlordiazepoxide, Cinolazepam, Clobazam, Clonazepam, Clotiazepam, Cloxazolam, Estazolam, Alprazolam, Ethyl loflazepate, Etizolam, Fludiazepam, Flunitrazepam, Flurazepam, Halazepam, Ketazolam, Loprazolam, Lormetazepam, Medazepam, Nitrazepam, Nordiazepam, Oxazepam, Pinazepam, Prazepam, Quazepam, Temazepam, Tofisopam, Triazolam, Flutazolam, Flutoprazepam, Nimetazepam, Mexazolam and Haloxazolam. More preferably, the benzodiazepine or analogue thereof is Diazepam.

In a preferred embodiment, the combination product comprises Salvinorin A and Medetomidine. In an alternative embodiment, the combination product comprises Salvinorin A and Diazepam.

In a preferred embodiment, the combination product is a composition or mixture of the KOR agonist, and the positive GABA_{A} receptor effector and/or α₂-adrenergic receptor agonist. In an alternative embodiment, the KOR agonist, and the positive GABA_{A} receptor effector and/or α₂-adrenergic receptor agonist are physically separated. For example, the KOR agonist could be contained in one blister pack while the positive GABA_{A} receptor effector or α₂-adrenergic receptor agonist is contained within a separate blister pack or the KOR agonist, and the positive GABA_{A} receptor effector or α₂-adrenergic receptor agonist could be contained in the same pill but be physically separated by a barrier, such as a gelatin barrier.

In a preferred embodiment, the combination product is contained within one or two tablets which further comprise common excipients and the tablet(s) is/are suitable for oral administration. The tablet(s) may comprise (i) the KOR agonist, and (ii) the positive GABA_{A} receptor effector and/or α₂-adrenergic receptor agonist, a first control-release coating comprising a water-insoluble water-permeable film-forming polymer, a plasticizer and a water-soluble polymer. The tablet(s) may further comprise a moisture barrier surrounding said first control-releasing coat, wherein the moisture barrier comprises an enteric polymer, a plasticizer and a permeation enhancer.

Non-limiting examples of water-insoluble water-permeable film-forming polymers useful for the control-releasing coat include cellulose ethers, cellulose esters, and polyvinyl alcohol. Non-limiting examples of plasticizers useful for the control-releasing coat described herein include polyols, such as polyethylene glycol of various molecular weights, organic esters, such as diethyl phthalate or triethyl citrate, and oils/glycerides such as fractionated coconut oil or castor oil. Non-limiting examples of water-soluble polymers useful for the control-releasing coat include polyvinylpyrrolidone, hydroxypropyl methylcellulose and hydroxypropyl cellulose. The preferred water-soluble polymer is polyvinylpyrrolidone. Non-limiting examples of enteric polymers useful for the moisture barrier include acrylic polymers such as a methacrylic acid copolymer type C [poly(methacrylic acid, methyl methacrylate) 1:1] available commercially under the trade name Eudragit® (e.g. Eudragit L 30 D-55). Non-limiting examples of permeation enhancers useful for the moisture barrier include silicon dioxide, colloidal silicon, lactose, hydrophilic polymers, sodium chloride, aluminum oxide, colloidal aluminum oxide, silica, microcrystalline cellulose and any combination thereof.

In a preferred embodiment, the aforementioned tablet(s) or any alternative tablet arrangement conceivable by a skilled person, e.g. such as a tablet formulation which keeps the KOR agonist, and the positive GABA_{A} receptor effector and/or α₂-adrenergic receptor agonist physically separated before administration, may be contained in one or more blister packs.

In a preferred embodiment, the combination product comprises a KOR agonist and instructions on how to administer the KOR agonist with a positive GABA_{A} receptor effector and/or α₂-adrenergic receptor agonist which may or may not be sold separately. In another preferred embodiment, the combination product comprises a positive GABA_{A} receptor effector and/or α₂-adrenergic receptor agonist and instructions on how to administer the positive GABA_{A} receptor effector and/or α₂-adrenergic receptor agonist with a KOR agonist which may or may not be sold separately.

In a preferred embodiment, the combination product may comprise one or more solution(s) which are suitable for intravenous, intramuscular, transdermal and/or subcutaneous administration. In another embodiment, the combination product may comprise one or more solution(s) which are suitable for sublingual, buccal and/or inhalation-mediated administration routes. In an alternative embodiment, the combination product may comprise one or more aerosol(s) which are suitable for inhalation-mediated administration.

In a preferred embodiment, the combination product may comprise one or more cream(s) and/or ointment(s) which are suitable for topical administration. In a preferred embodiment, the combination product may comprise one or more suppositories which are suitable for rectal administration.

The combination product may comprise any combination of tablets, solutions, aerosols, creams, ointments and/or suppositories as long as the combination product induces or maintains general anesthesia in a subject or animal.

Pharmaceutical Composition

In a second aspect, the present invention provides a pharmaceutical composition comprising the combination product of the present invention and a pharmaceutically acceptable carrier, pharmaceutically acceptable diluent and/or pharmaceutically acceptable excipient.

A pharmaceutical composition as described herein may also contain other substances. These substances include, but are not limited to, cryoprotectants, lyoprotectants, surfactants, bulking agents, anti-oxidants, and stabilizing agents. In some embodiments, the pharmaceutical composition may be lyophilized.

The term “cryoprotectant” as used herein, includes agents which provide stability to the combination product against freezing-induced stresses. Cryoprotectants may also offer protection during primary and secondary drying and long-term product storage. Non-limiting examples of cryoprotectants include sugars, such as sucrose, glucose, trehalose, mannitol, mannose, and lactose; polymers, such as dextran, hydroxyethyl starch and polyethylene glycol; surfactants, such as polysorbates (e.g., PS-20 or PS-80); and amino acids, such as glycine, arginine, leucine, and serine. A cryoprotectant exhibiting low toxicity in biological systems is generally used.

In one embodiment, a lyoprotectant is added to a pharmaceutical composition described herein. The term “lyoprotectant” as used herein, includes agents that provide stability to the combination product during the freeze-drying or dehydration process (primary and secondary freeze-drying cycles. This helps to minimize product degradation during the lyophilization cycle, and improve the long-term product stability. Non-limiting examples of lyoprotectants include sugars, such as sucrose or trehalose; an amino acid, such as monosodium glutamate, non-crystalline glycine or histidine; a methylamine, such as betaine; a lyotropic salt, such as magnesium sulfate; a polyol, such as trihydric or higher sugar alcohols, e.g., glycerin, erythritol, glycerol, arabitol, xylitol, sorbitol, and mannitol; propylene glycol; polyethylene glycol; pluronics; and combinations thereof. The amount of lyoprotectant added to a pharmaceutical composition is generally an amount that does not lead to an unacceptable amount of degradation when the pharmaceutical composition is lyophilized.

In some embodiments, a bulking agent is included in the pharmaceutical composition. The term “bulking agent” as used herein, includes agents that provide the structure of the freeze-dried product without interacting directly with the pharmaceutical product. In addition to providing a pharmaceutically elegant cake, bulking agents may also impart useful qualities in regard to modifying the collapse temperature, providing freeze-thaw protection, and enhancing the stability over long-term storage. Non-limiting examples of bulking agents include mannitol, glycine, lactose, and sucrose. Bulking agents may be crystalline (such as glycine, mannitol, or sodium chloride) or amorphous (such as dextran, hydroxyethyl starch) and are generally used in formulations in an amount from 0.5% to 10%.

Other pharmaceutically acceptable carriers, excipients, or stabilizers, such as those described in Remington's Pharmaceutical Sciences 16th edition, Osol, A. Ed. (1980) or Remington: The Science and Practice of Pharmacy 22^{nd} edition, Pharmaceutical press (2012), ISBN-13: 9780857110626 may also be included in a pharmaceutical composition described herein, provided that they do not adversely affect the desired characteristics of the pharmaceutical composition.

For solid pharmaceutical compositions, conventional nontoxic solid carriers may be used which include, for example, pharmaceutical grades of mannitol, lactose, starch, magnesium stearate, sodium saccharin, talcum, cellulose, glucose, sucrose, magnesium carbonate, and the like. For solution for injection, the pharmaceutical composition may further comprise cryoprotectants, lyoprotectants, surfactants, bulking agents, anti-oxidants, stabilizing agents and pharmaceutically acceptable carriers. For aerosol administration, the pharmaceutical compositions are generally supplied in finely divided form along with a surfactant and propellant. The surfactant must, of course, be nontoxic, and is generally soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. A carrier can also be included, as desired, as with, e.g., lecithin for intranasal delivery. For suppositories, traditional binders and carriers may include, for example, polyalkalene glycols or triglycerides.

Medical Uses

In a third aspect, the present invention provides the combination product of the present invention or the pharmaceutical composition of the present invention for use as a medicament. In a fourth aspect, the present invention provides the combination product of the present invention or the pharmaceutical composition of the present invention for use in the induction and/or maintenance of general anesthesia in a subject or animal.

In a preferred embodiment, the combination product of the present invention or pharmaceutical composition of the present invention is administered continuously or discontinuously. For example, the patient may be administered the combination product or pharmaceutical composition via continuous intravenous infusion or the patient may be administered the combination product or pharmaceutical composition through several discrete injections.

In a preferred embodiment, the κ-opioid receptor agonist, and the α₂-adrenergic receptor agonist and/or positive GABA_{A} receptor effector are administered together or separately. For example, Salvinorin A and Diazepam were administered together in a single injection to Rat number 3 of Example 4 and Salvinorin A and Medetomidine were administered separately to Rat number 3 of Example 3.

In a preferred embodiment, the combination product or pharmaceutical composition of the present invention is administered intravenously, intraperitoneally or via inhalation. Where the combination product or pharmaceutical composition is administered via inhalation, the combination product or pharmaceutical composition may be aerosolized and administered via an anesthesia mask.

In a preferred embodiment, the α₂-adrenergic receptor agonist and/or positive GABA_{A} receptor effector is administered first and then the κ-opioid receptor agonist is administered. For example, this was done to Rat number 3 of Examples 3 and 4 of the present application. This approach is common in veterinary medicine and has the advantage that the animal is first sedated which facilitates their manipulation.

In a preferred embodiment, the κ-opioid receptor agonist and the positive GABA_{A} receptor effector is administered at a mass ratio of at least 1:1 and/or the κ-opioid receptor agonist and the α₂-adrenergic receptor agonist is administered at a mass ratio of at least 20:1. Preferably, the κ-opioid receptor agonist and the positive GABA_{A} receptor effector is administered at a mass ratio of at least 1:1, 2:1, 3:1, 4:1 or 5:1, and/or the κ-opioid receptor agonist and the α₂-adrenergic receptor agonist is administered at a mass ratio of at least 20:1, 40:1, 60:1, 80:1 or 100:1. More preferably, the κ-opioid receptor agonist and the positive GABA_{A} receptor effector is administered at a mass ratio of at least 6:1 and/or the κ-opioid receptor agonist and the α₂-adrenergic receptor agonist is administered at a mass ratio of at least 120:1.

A selective κ-opioid receptor agonist for use in a method of inducing or maintaining a state of general anesthesia in a subject or animal, wherein the selective κ-opioid receptor agonist is co-administered with a α₂-adrenergic receptor agonist and/or a positive GABA_{A} receptor effector is encompassed by the present invention. A α₂-adrenergic receptor agonist and/or a positive GABA_{A} receptor effector for use in a method of inducing or maintaining a state of general anesthesia in a subject or animal, wherein the α₂-adrenergic receptor agonist and/or the positive GABA_{A} receptor effector is co-administered with a selective κ-opioid receptor agonist is also encompassed by the present invention.

Kit

In a fifth aspect, the present invention provides a kit comprising the combination product of the present invention, and a pharmaceutically acceptable carrier, a pharmaceutically acceptable diluent and/or a pharmaceutically acceptable excipient. Any of the carriers, diluents and excipients described in the present application may be included in the kit. Further, any embodiment of the combination product may be included in the kit. Thus, the kit may comprise a selective κ-opioid receptor agonist and, a α₂-adrenergic receptor agonist and/or positive GABA_{A} receptor effector or the kit may, for example, only comprise a selective κ-opioid receptor agonist and instructions on how to administer the KOR agonist with a positive GABA_{A} receptor effector and/or α₂-adrenergic receptor agonist.

In a sixth aspect, the present invention provides the use of the kit for the manufacture of a general anesthetic. The general anesthetic may be used to induce and/or maintain general anesthesia in a subject or animal.

Items

In some embodiments, the present application provides the following items:

[1] A combination product comprising:

(i) one or more selective κ-opioid receptor agonists, preferably a diterpene or diterpenoid compound, more preferably a clerodane diterpene or clerodane diterpenoid compound; and

(ii) one or more positive GABA_{A} receptor effectors and/or one or more α₂-adrenergic receptor agonists.

[2] The combination product according to item [1], wherein the selective κ-opioid receptor agonist is Salvinorin A or B, or analogue thereof, preferably one or more κ-opioid receptor agonists selected from Table 2.

[3] The combination product according to item [1] or [2] wherein:

(a) the α₂-adrenergic receptor agonist is selected from a group consisting of Medetomidine, Dexmedetomidine, Romifidine, Detomidine, Xylazine, Clonidine, Agmatine, Lofexidine, Tizanidine, Guanfacine, Guanabenz and Mivazerol; and/or

(b) the positive GABA_{A} receptor effector is selected from a group consisting of Diazepam, Midazolam, Lorazepam, Zolazepam, Etomidate, Adinazolam, Bentazepam, Bromazepam, Brotizolam, Camazepam, Chlorazepam, Chlordiazepoxide, Cinolazepam, Clobazam, Clonazepam, Clotiazepam, Cloxazolam, Estazolam, Alprazolam, Ethyl loflazepate, Etizolam, Fludiazepam, Flunitrazepam, Flurazepam, Halazepam, Ketazolam, Loprazolam, Lormetazepam, Medazepam, Nitrazepam, Nordiazepam, Oxazepam, Pinazepam, Prazepam, Quazepam, Temazepam, Tofisopam, Triazolam, Flutazolam, Flutoprazepam, Nimetazepam, Mexazolam, Haloxazolam, Desflurane, Enflurane, Halothane, Isoflurane, Methoxyflurane, Nitrous oxide, Sevoflurane, Thiopental, Thiopental sodium salt, Thiamylal, Pentobarbital, Secobarbital, Barbital, Methohexital, Chloral, Zaleplon, Zolpidem, Zopiclone, Eszopiclone, Desmetilzopiclone, Indiplon, Chloral hydrate, Triclofos, and Triclofos sodium salt.

[4] The combination product according to any one of items [1] to [3], wherein the combination product is prepared for oral, sublingual, buccal, intranasal, intravenous, intramuscular, intraperitoneal and/or inhalation-mediated administration.

[5] The combination product according to any one of items [1] to [4], wherein:

(a) the combination product is a composition; or

(b) the one or more selective κ-opioid receptor agonists, and the one or more α₂-adrenergic receptor agonists and/or one or more positive GABA_{A} receptor effectors are physically separated.

[6] A pharmaceutical composition comprising the combination product according to item [5](a) and a pharmaceutically acceptable carrier, a pharmaceutically acceptable diluent and/or a pharmaceutically acceptable excipient.

[7] The combination product according to any one of items [1] to [5] or the pharmaceutical composition according to item [6] for use as a medicament.

[8] The combination product according to any one of items [1] to [5] or the pharmaceutical composition according to item [6] for use in the induction and/or maintenance of general anesthesia in a subject or animal.

[9] The combination product or pharmaceutical composition for use according to item [8], wherein the combination product or pharmaceutical composition is administered continuously or discontinuously.

[10] The combination product or pharmaceutical composition for use according to item [8] or [9], wherein the κ-opioid receptor agonist, and the α₂-adrenergic receptor agonist and/or positive GABA_{A} receptor effector are administered together or separately.

[11] The combination product or pharmaceutical composition for use according to any one of items [8] to [10], wherein the combination product or pharmaceutical composition is administered intravenously, intraperitoneally or via inhalation.

[12] The combination product or pharmaceutical composition for use according to item [8], [9] or [11], wherein the α₂-adrenergic receptor agonist and/or positive GABA_{A} receptor effector is administered first and then the κ-opioid receptor agonist is administered.

[13] The combination product or pharmaceutical composition for use according to any one of items [8] to [12], wherein the κ-opioid receptor agonist and the positive GABA_{A} receptor effector is administered at a mass ratio of at least 6:1 and/or the κ-opioid receptor agonist and the α₂-adrenergic receptor agonist is administered at a mass ratio of at least 120:1.

[14] A kit comprising:

(i) the combination product according to any one of items [1] to [5]; and

(ii) a pharmaceutically acceptable carrier, a pharmaceutically acceptable diluent and/or a pharmaceutically acceptable excipient.

[15] Use of the kit according to item [14] for the manufacture of a general anesthetic.

[16] A selective κ-opioid receptor agonist, preferably a diterpene or diterpenoid compound, more preferably a clerodane diterpene or clerodane diterpenoid compound, for use in a method of inducing or maintaining a state of general anesthesia in a subject or animal, wherein the selective κ-opioid receptor agonist is co-administered with a α₂-adrenergic receptor agonist and/or a positive GABA_{A} receptor effector.

[17] A α₂-adrenergic receptor agonist and/or a positive GABA_{A} receptor effector for use in a method of inducing or maintaining a state of general anesthesia in a subject or animal, wherein the α₂-adrenergic receptor agonist and/or the positive GABA_{A} receptor effector is co-administered with a selective κ-opioid receptor agonist, preferably a diterpene or diterpenoid compound, more preferably a clerodane diterpene or clerodane diterpenoid compound.

### EXAMPLES

In all four examples provided below, the administered Salvinorin A (SA; CAS No. 83729-01-5) was purchased from THC-Pharm GmbH, Frankfurt, Germany (catalog No. 1152). SA solutions for parenteral administration were prepared using dimethyl sulfoxide as vehicle (DMSO; CAS No. 67-68-5). DMSO was purchased from Acofarma S.A, Tarrassa (Barcelona), Spain (catalog No. 1126051). All vehicle and drug injections were administered as a bolus.

To assess the presence of sedation, unconsciousness, general anesthesia and analgesia, a series of observations and tests were conducted. These were adapted from commonly used procedures (Eger et al., 1965; Gustafsson et al., 1996):

Decreases in spontaneous locomotor and/or exploratory activity: Immediately after administration of each bolus injection, the animal was assessed for any changes in spontaneous locomotor and/or exploratory activity. If these behaviors were found to be decreased as compared to the pre-administration state, it was considered that sedation had been induced. If the spontaneous righting reflex (see here below) had not been lost after dosing but sedation was observed, the animal was monitored at regular intervals to assess the duration of the induced sedation.

The spontaneous righting reflex: Before and at a series of set time points following each bolus injection, the animal was placed on its back and righting time measured. Righting normally occurs under 15 seconds. Whenever the animal was unable to right itself, it was considered that the spontaneous righting reflex had been lost and unconsciousness induced.

The provoked righting reflex: This reflex was assessed whenever the spontaneous righting reflex was not present. The experimenter applied a nociceptive pressure stimulus (a manual pinch) to the hind paw. If the animal was unable to right itself following this stimulus, it was considered that the provoked righting reflex had been lost and the animal had reached the state of general anesthesia (unconsciousness and lack of arousal following painful stimuli).

Response to nociceptive stimulation: Following the loss of the provoked righting reflex, the animal was kept resting on its back and the same nociceptive stimulus (manual pinch) was applied to the same hind paw at regular intervals until any sudden movement of the paw, head or body of the animal (without righting) was detected. At this point, no further assessments were conducted, and the animal was monitored until the spontaneous righting reflex was recovered. The absence of movement to the nociceptive stimulus was considered as an indicator of effective analgesia within the general anesthesia state.

Duration of unconsciousness: The time lapse since the loss of the spontaneous righting reflex until its recovery was recorded and considered a measurement of the overall duration of unconsciousness.

### Example 1

### Salvinorin A Administered Alone Intraperitoneally does not Induce General Anesthesia

Three male Sprague Dawley rats were used in this example. All three showed similar levels of spontaneous activity before the interventions described below. In order to test SA, two different solutions of SA in DMSO were prepared. The first at a concentration of 12 mg/ml and the second at 24 mg/ml.

Rat number 1 (weight 0.347 kg) served as control and received an intraperitoneal (i.p.) injection of 0.2 ml of DMSO vehicle. No changes in locomotor or exploratory activity that could suggest sedation were observed immediately after the injection. Sedation and the spontaneous righting reflex were assessed at 1, 2, 5, 10, 15, 20, 30 and 40 minutes after the injection. As described above, to assess the spontaneous righting reflex the rat is put on its back and the time taken until the animal stands again on its four limbs is measured. At all measurement points the rat resisted being turned on its back and fought actively to recover its natural position. Righting was achieved within 1-2 seconds. The administered DMSO vehicle had no effect on spontaneous locomotor or exploratory activity, nor on the spontaneous righting reflex. Consequently, no sedation or general anesthesia were observed after DMSO administration.

Rat number 2 (weight 0.375 kg) received an 0.2 ml i.p. injection of the SA in DMSO solution prepared at the lower 12 mg/ml concentration. Thus, the total SA dose administered was 2.4 mg, equivalent to 6.4 mg/kg. Similar to Rat number 1, Rat number 2 did not show any changes in locomotor or exploratory activity immediately after the injection. At all assessment time points (the same used for Rat number 1), Rat number 2 resisted being turned on its back and righting was achieved also within 1-2 seconds. No effect was therefore observed on the spontaneous righting reflex. However, between 10 and 20 minutes after the injection, Rat number 2 showed decreased locomotor and exploratory activity as compared to the pre-drug state. At 30 minutes post-injection, locomotor and exploratory activity were comparable to baseline levels. In conclusion, light sedation was observed at the 6.4 mg/kg SA dose, but no loss of consciousness or general anesthesia.

Rat number 3 (weight 0.372 kg) received an 0.2 ml i.p. injection of the SA in DMSO solution prepared at the higher 24 mg/ml concentration. Thus, the total SA dose administered was 4.8 mg, equivalent to 12.9 mg/kg. Similar to Rats number 1 and number 2, Rat number 3 did not show any changes in locomotor or exploratory activity immediately after the injection. At all assessment time points (the same used for Rats number 1 and 2), Rat number 3 resisted being turned on its back and righting was achieved also within 1-2 seconds. Again, no effect was observed on the the spontaneous righting reflex. However, between 10 and 25 minutes after the injection, Rat number 3 showed decreased locomotor and exploratory activity as compared to the pre-drug state. The decrease was qualitatively similar in intensity to that seen for Rat number 2, but of longer duration. At 30 minutes, locomotor and exploratory activity had increased, and at 40 minutes these behaviors were comparable to baseline levels. In conclusion, slight sedation of longer duration was observed at the 12.9 mg/kg SA dose, but no loss of consciousness or general anesthesia.

Example 1 demonstrates that: a) General anesthesia was not achieved at any of the two SA i.p. doses administered. The 12.9 mg/kg high dose was larger than the highest administered dose (10 mg/kg) found in the literature for the same animal species and administration route (Wang et al., 2008; Teksin et al., 2009); b) At the high 12.9 mg/kg dose, the i.p. administration of SA alone induced only light sedation but no loss of consciousness or general anesthesia.

### Example 2

### Salvinorin A Administered Alone Intravenously does not Induce General Anesthesia

Three male Sprague Dawley rats were used in this example. All three showed similar levels of spontaneous activity before the interventions described below. In order to test SA, two different solutions of SA in DMSO were prepared. The first at a concentration of 12 mg/ml and the second at 24 mg/ml. Vehicle and drug solutions were administered by intravenous injection (i.v.) in the tail, with the animal placed in a standard cylindrical restrainer. Prior to injection, the tail was submerged in warm (30-35° C.) water for 1-2 minutes until adequate dilation of the tail veins was achieved.

Rat number 1 (weight 0.333 kg) served as control and received an i.v. injection of 0.2 ml DMSO vehicle. No changes in locomotor or exploratory activity that could suggest sedation were observed immediately after the injection. Sedation and the spontaneous righting reflex were assessed at 1, 2, 5, 10, 15, 20, 30 and 40 minutes after the injection, as described in Example 1. At all measurement time points the rat resisted being turned on its back and fought to recover its natural position. Righting was achieved within 1-2 seconds. The administered DMSO vehicle had no effect on spontaneous locomotor or exploratory activity, nor on the on the spontaneous righting reflex. Consequently, no sedation, loss of consciousness or general anesthesia were observed.

Rat number 2 (weight 0.390 kg) received an 0.2 ml i.v. injection of the SA in DMSO solution prepared at the lower 12 mg/ml concentration. Thus, the total SA dose administered was 2 4 mg, equivalent to 6.2 mg/kg. Similar to Rat number 1, Rat number 2 did not show any changes in locomotor or exploratory activity immediately after the injection. At 10 minutes after the injection, locomotor and exploratory activity were decreased. The rat could be turned on its back, recovering its natural position (spontaneous righting reflex) in 8 seconds. This was higher than the 1-2 second baseline value. Thus, compared to Rat number 1, Rat number 2 showed increased sedation. At 15 minutes after the injection, Rat number 2 could not be turned on its back, but spontaneous locomotor and exploratory activity were still decreased. Locomotor activity remained decreased at 20 and 25 minutes. At 30 minutes post-injection, locomotor and exploratory activity were comparable to pre-injection values. In conclusion, at the 6.2 mg/kg SA dose only mild sedation was observed, but no loss of consciousness or general anesthesia.

Rat number 3 (weight 0.400 kg) received an 0.2 ml i.v. injection of the SA in DMSO solution prepared at the higher 24 mg/ml concentration. The total SA dose administered was 4.8 mg, equivalent to 12.0 mg/kg. Similar to Rats number 1 and number 2, Rat number 3 did not show any changes in locomotor or exploratory activity immediately after the injection. However, at 5 minutes after the injection, locomotor activity was decreased, the rat could be turned on its back, and recovered its natural position (spontaneous righting reflex) in 12 seconds. This was higher than the 1-2 second baseline value. Thus, compared to Rat number 1, Rat number 3 showed increased sedation. At 10 minutes after the injection, Rat number 3 could not be turned on its back, but spontaneous locomotor and exploratory activity were still decreased. These behaviors remained decreased at 15, 20 and 25 minutes after dosing. At 30 minutes post-injection, locomotor and exploratory activity were comparable to pre-injection values. In conclusion, at the 12.0 mg/kg SA dose only mild sedation was observed, but no loss of consciousness or general anesthesia. Qualitatively and in duration, the degree of sedation did not appear to be different from that induced by the half the dose in Rat number 2.

Example 2 demonstrates that: a) General anesthesia was not achieved at any of the two i.v. doses of SA administered. Only one study has reported the i.v. administration SA to rodents. A 1.8 mg/kg dose was administered to rats (between 3 and 6 times lower than our doses). However, the animals were under anesthesia and consequently no comparisons can be made in terms of behavioral impact (Placzek et al., 2015). On the other hand, i.v. SA has been administered to Rhesus monkeys at the maximum dose of 0.1 mg/kg, inducing sedative effects (Butelman et al., 2009). The 6.2 and 12 mg/kg doses administered here are 62 and 120 larger and, again, only mild sedative effects were observed; b) Even when administered i.v. (100% bioavailability), the effects of SA were not different from those following i.p. administration. As found for the i.p. administration route, SA alone only induced mild sedation, but did not induce loss of consciousness or general anesthesia.

### Example 3

### Salvinorin A Administered Intravenously in Combination with an Alpha-2-Adrenergic Agonist Induces Rapid and Dose-Dependent General Anesthesia

Three male Sprague Dawley rats were used in this example. All three showed similar levels of spontaneous activity before the interventions described below. In this example, SA was administered in association with an alpha-2-adrenergic agonist. Both drugs were injected intravenously (i.v.) in the tail. Medetomidine (CAS No. 86347-14-0) was the alpha-2-adrenergic agonist used. The i.v. injections of Medetomidine and SA were administered with the rat placed in a restrainer and following the same procedure described in Example 2. Rat number 1 received a single i.v. medetomidine injection. Rats number 2 and 3 received two consecutive i.v. injections. Different tail veins were used for the first (Medetomidine) and the second (SA) injections. The two injections were 20 minutes apart. A commercially available Medetomidine hydrochloride solution was used (Domtor®, Orion Corporation, Espoo, Finland). The original 1 mg/ml Domtor® solution was diluted in saline to a final concentration of 0.1 mg/ml. Two different SA solutions in DMSO were prepared. The first at a concentration of 12 mg/ml and the second at 24 mg/ml.

Rat number 1 (weight 0.200 kg) served as control and received an 0.1 ml i.v. injection of the 0.1 mg/ml medetomidine hydrochloride solution. The total dose administered was 0.010 mg (10 μg), equivalent to 0.050 mg/kg. A decrease in spontaneous locomotor and exploratory activity indicating sedation was observed at 30 seconds after the injection. Sedation lasted until 30 minutes after the injection. The spontaneous righting reflex was assessed at 1, 2, 5, 10, 15, 20, 30 and 40 minutes after the injection, as described in Example 1. At all assessment points, the rat resisted being turned on its back and righting was achieved within 2-3 seconds. Thus, the spontaneous righting reflex was preserved throughout the experimental session. The administered medetomidine dose had sedative effects but did not induce loss of consciousness or general anesthesia.

Rat number 2 (weight 0.195 kg) received an 0.1 ml i.v. injection of the 0.1 mg/ml medetomidine hydrochloride solution. The total administered dose was 0.010 mg (10 μg), equivalent to 0.051 mg/kg. As observed for Rat number 1, a rapid sedative effect was observed after the medetomidine injection, as reflected by the reduction in spontaneous locomotor and exploratory activity at 30 seconds. A 20-minute waiting period was kept between the medetomidine and Salvinorin A injections. The spontaneous righting reflex remained preserved at 1, 2, 5, 10, 15 and 20 minutes after medetomidine.

At all 6 time-points, righting was achieved within 2-3 seconds. At 20 min the second i.v. injection containing SA was administered. The injection contained 0.1 ml of the 12 mg/ml solution of SA in DMSO. The total SA dose administered was 1.2 mg, equivalent to 6.2 mg/kg. At 30 seconds after the SA injection the spontaneous righting reflex was abolished (i.e. unconsciousness achieved) and the rat lay motionless resting on its back. At 2 minutes after the SA injection, the provoked righting reflex (manual pinch to a hind paw) was lost, indicating that general anesthesia had been achieved. The rat did not elicit any movements when administered the same nociceptive stimulus (manual pinch) at 3, 4, 5 and 6 minutes post-injection. At 7 minutes, the first reaction to nociceptive stimulation (paw withdrawal) was observed and no further assessments were conducted. At 20 min after SA administration, the rat recovered the spontaneous righting reflex, turning itself and regaining its normal standing position. In conclusion, the combination of 0.051 mg/kg medetomidine with 6.2 mg/kg SA in two consecutive i.v. injections rapidly induced unconsciousness and general anesthesia after the SA dose. Analgesia was maintained for 5 minutes and the overall duration of unconsciousness was 19 minutes.

Rat number 3 (weight 0.205 kg) received an 0.1 ml i.v. injection of the 0.1 mg/ml medetomidine hydrochloride solution. The total administered dose was 0.010 mg (10 μg), equivalent to 0.049 mg/kg. As observed for Rats number 1 and 2, spontaneous locomotor and exploratory activity rapidly decreased, indicating a sedative effect. During the 20-minute waiting period, the spontaneous righting reflex remained preserved. At 1, 2, 5, 10, 15 and 20 minutes, righting was achieved within 2-3 seconds. At 20 min, the second i.v. injection containing SA was administered. A volume of 0.1 ml of the 24 mg/ml solution of SA in DMSO was injected. This corresponded to a total SA dose of 2.4 mg, equivalent to 11.7 mg/kg. The rat was motionless immediately after the end of the injection and lost the spontaneous righting reflex (i.e. unconsciousness achieved). The provoked righting reflex (manual pinch to a hind paw) was absent at the first assessment point 1 minute after the SA injection, indicating that general anesthesia had been achieved. The rat did not react to the nociceptive pinching stimulus applied to a hind paw at 2, 5, 10, 15, 20 and 25 minutes. At 30 minutes, pinching led to a sudden withdrawal movement, indicating a reaction to painful stimulation. No further analgesia assessments were conducted. At 56 min after SA administration, the rat recovered the spontaneous righting reflex, turning itself and regaining its normal standing position. In conclusion, the combination of 0.051 mg/kg medetomidine with 11.7 mg/kg SA in two consecutive i.v. injections produced immediate unconsciousness and rapid general anesthesia after SA administration. Effects were faster and more prolonged at the 11.7 mg/kg dose, as compared to the lower 6.2 mg/kg dose. Analgesia was maintained for 29 minutes and the overall duration of unconsciousness was 56 minutes. Example 3 demonstrates that: a) General anesthesia was achieved by combining an i.v. dose of an alpha-2-adrenergic agonist with i.v. doses of SA in the 6-12 mg/kg dose range. This effect had not observed when SA was administered alone by i.v. injection in the same dose range (Example 2). It was also absent when only the alpha-2-adrenergic agonist was administered (Example 3, Rat number 1); b) The state induced by the drug combination presented the three defining elements of general anesthesia, i.e., loss of consciousness, lack of movements (voluntary and reflex), and lack of response to painful stimuli (analgesia); c) Induction speed, duration of analgesia and overall duration of unconsciousness were dose dependent, with more intense effects observed after the high SA dose.

### Example 4

### Salvinorin A Administered Intravenously in Combination with a Positive Effector of the GABA-A Receptor (Diazepam) Induces Rapid and Dose-Dependent General Anesthesia

Three male Sprague Dawley rats were used in this example. All three showed similar levels of spontaneous activity before the interventions described below. In this example, SA was administered in association with a positive effector of the GABA-A receptor, i.e., a benzodiazepine with positive allosteric modulator activity at this site. Both drugs were administered intravenously (i.v.) in the tail. Diazepam (CAS No. 439-14-5) was the benzodiazepine used. The i.v. injections of Diazepam and SA were administered with the rat placed in a restrainer and following the procedure described in Examples 2 and 3. Rat number 1 received a single i.v. Diazepam injection. Rats number 2 and 3 received two consecutive i.v. injections. Different tail veins were used for the first injection (Diazepam) and the second (SA). The two injections were 10 minutes appart. A commercially availabe Diazepam solution was used (Ziapam®, Laboratoire TVM, Lempdes, France). The original 5 mg/ml Ziapam® was diluted in 96% ethanol to a final concentration of 2.5 mg/ml. A single solution of SA in DMSO was prepared at a concentration of 14 mg/ml.

Rat number 1 (weight 0.255 kg) served as control and received an 0.1 ml i.v. injection of the 2.5 mg/ml diazepam solution. The total dose administered was 0.250 mg, equivalent to 0.98 mg/kg. A decrease in spontaneous locomotor and exploratory activity indicating sedation was observed immediately after the end of the injection. Spontaneous locomotor and exploratory activity was partially recovered at 30 minutes. Full recovery with normal spontaneous locomotor and exploratory activity was observed at 1 h. The spontaneous righting reflex was assessed at 1, 2, 5, 10, 15, 20, 30, 40, 50 and 60 minutes after the injection, as described in Example 1. The reflex was not lost in the course of the 60-minute observation period. At all assessment points, the rat resisted being turned on its back and righting was achieved within 2-3 seconds. The administered diazepam dose had sedative effects but did not induce loss of consciousness or general anesthesia.

Rat number 2 (weight 0.252 kg) received an 0.1 ml i.v. injection of the 2.5 mg/ml diazepam solution. The total dose administered was 0.250 mg, equivalent to 0.99 mg/kg. As observed for Rat number 1, clear sedation appeared immediately after the diazepam injection, characterized by a marked decrease in the rat's spontaneous locomotor and exploratory activity. A 10-minute waiting period was established between the diazepam and Salvinorin A injections. The spontaneous righting reflex remained preserved at 1, 2, 5 and 10 minutes after diazepam. Although sedated, the rat resisted being put on its back at all four assessment time points and righting was achieved within 2-3 seconds. At 10 min, the rat received the second i.v. injection containing 0.110 ml of the 14 mg/ml solution of SA in DMSO. The administered SA dose was 1.54 mg, equivalent to 6.1 mg/kg. At 1 min after the SA injection the rat had lost the spontaneous righting reflex, resting motionless when the experimenter put it on its back (i.e., unconsciousness achieved). At 2 minutes after the SA injection, the provoked righting reflex (manual pinch to a hind paw) was lost, indicating that general anesthesia had been achieved. The rat did not respond to the nociceptive stimulus to the paw at 5, 10, 15 and 20 minutes after the injection. The first reaction to nociceptive stimulation was observed 25 minutes after the SA injection. No further assessments of analgesia were conducted from this point onwards. At 35 min after SA administration, the rat recovered the spontaneous righting reflex, turning itself and regaining its normal standing position. In conclusion, the combination of 0.99 mg/kg diazepam with 6.1 mg/kg SA in two consecutive i.v. injections rapidly led to unconsciousness and general anesthesia after the SA dose. Analgesia was maintained for 23 minutes and the overall duration of unconsciousness was 34 minutes.

Rat number 3 (weight 0.245 kg) received the same total amount of diazepam (0.250 mg; 1.02 mg/kg), but split between the first and second injections. The first i.v. injection contained 0.05 ml of the 2.5 mg/ml diazepam solution. The total dose administered in this first injection was 0.125 mg, equivalent to 0.51 mg/kg. At 1 minute, a decrease in spontaneous locomotor and exploratory activity was observed. Sedation was qualitatively milder than that observed for Rats number 1 and 2. The spontaneous righting reflex remained preserved at 1, 2, 5 and 10 minutes after diazepam. Righting was achieved within 2-3 seconds. At 10 minutes, the second i.v. injection was administered containing a mixture of 0.05 ml of the 2.5 mg/ml diazepam solution, and 0.125 ml of the 14 mg/ml solution of SA in DMSO. The total administered volume was 0.175 ml. The administered diazepam dose was 0.125 mg, equivalent to 0.51 mg/kg (the same as in the first injection). The SA dose was 1.75 mg, equivalent to 7.1 mg/kg. The rat was motionless immediately after the end of the second injection containing SA+Diazepam, and lost the spontaneous righting reflex (i.e. unconsciousness achieved). The provoked righting reflex (manual pinch to a hind paw) was absent at the first assessment point 1 minute after the SA+Diazepam injection, indicating that general anesthesia had been achieved. The rat did not react to the nociceptive stimulus at 2, 5, 10, 15, 20, 25 and 30 minutes. The first response was seen at 35 minutes after the SA+Diazepam injection. No further analgesia assessments were conducted. At 50 min after SA administration, the rat recovered the spontaneous righting reflex, turning itself and regaining its normal standing position.

In conclusion, general anesthesia was again effectively achieved with a diazepam and SA combination. Importantly, immediate unconsciousness and rapid general anesthesia were achieved with the same total diazepam dose employed for Rat number 2 but split between the first and second injections. Analgesia was maintained for 23 minutes and the overall duration of unconsciousness was 34 minutes. Compared with the dose used and the results obtained for Rat number 2, the administration regime used for Rat number 3 with a 16% increase in SA dose (from 6.1 to 7.1 mg/kg), led to a 52% increase the duration of analgesia (from 23 to 35 minutes), and to a 47% increase in the overall duration of unconsciousness (from 34 to 50 minutes).

Example 4 demonstrates that: a) General anesthesia was achieved by combining i.v. SA in the 6-7 mg/kg dose range with a positive effector of the GABA-A receptor (the benzodiazepine diazepam, a positive allosteric modulator). This is in clear contrast with the results from Example 2, where SA was administered alone by i.v. injection at a dose 69% higher (12 mg/kg); b) The state induced by the drug combination presented the defining elements of general anesthesia, i.e., loss of consciousness, lack of movements (voluntary and reflex), and lack of response to painful stimuli (analgesia); c) Immediate loss of consciousness and prolonged general anesthesia was obtained with the administration of an i.v. formulation containing a mixture of SA and the benzodiazepine in a single injection. This injection was an effective general anesthetic when administered to an animal that had received 50% less pre-anesthetic sedation with diazepam; d) The formulation containing the mixture of SA and the benzodiazepine in a single injection attained larger increases in the duration of analgesia and unconsciousness than could be expected by the small increase in the SA dose administered. The synergistic effect attained by the formulation could be used to decrease the total SA dose administered to the subject and consequently any potential SA-related untoward events.

### NON-PATENT LITERATURE REFERENCES

American Society of Anesthesiologists (2014) Continuum of depth of sedation, deinition of general anesthesia and levels of sedation/analgesia. Available at: www.asahq.org.

Ansonoff M A, Zhang J, Czyzyk T, Rothman R B, Stewart J, Xu H, Zjwiony J, Siebert D J, Yang F, Roth B L, Pintar J E (2006) Antinociceptive and hypothermic effects of Salvinorin A are abolished in a novel strain of kappa-opioid receptor-1 knockout mice. J Pharmacol Exp Ther 318:641-648.

Bailey P L, Wilbrink J, Zwanikken P, Pace N L, Stanley T H (1985) Anesthetic induction with fentanyl. Anesth Analg 64:48-53.

Beerepoot P, Lam V, Luu A, Tsoi B, Siebert D, Szechtman H (2008) Effects of salvinorin A on locomotor sensitization to D2/D3 dopamine agonist quinpirole. Neurosci Lett 446:101-104.

Béguin C, Richards M R, Wang Y, Chen Y, Liu-Chen L-Y, Ma Z, Lee D Y W, Carlezon W A, Cohen B M (2005) Synthesis and in vitro pharmacological evaluation of salvinorin A analogues modified at C(2). Bioorg Med Chem Lett 15:2761-2765.

Béguin C, Richards M R, Li J-G, Wang Y, Xu W, Liu-Chen L-Y, Carlezon W A, Cohen B M (2006) Synthesis and in vitro evaluation of salvinorin A analogues: effect of configuration at C(2) and substitution at C(18). Bioorg Med Chem Lett 16:4679-4685.

Béguin C, Potter D N, Dinieri J A, Munro T A, Richards M R, Paine T A, Berry L, Zhao Z, Roth B L, Xu W, Liu-Chen L-Y, Carlezon W A, Cohen B M (2008) N-methylacetamide analog of salvinorin A: a highly potent and selective kappa-opioid receptor agonist with oral efficacy. J Pharmacol Exp Ther 324:188-195.

Béguin C, Duncan K K, Munro T A, Ho D M, Xu W, Liu-Chen L-Y, Carlezon W A, Cohen B M (2009) Modification of the furan ring of salvinorin A: identification of a selective partial agonist at the kappa opioid receptor. Bioorg Med Chem 17:1370-1380.

Béguin C, Potuzak J, Xu W, Liu-Chen L-Y, Streicher J M, Groer C E, Bohn L M, Carlezon W A, Cohen B M (2012) Differential signaling properties at the kappa opioid receptor of 12-epi-salvinorin A and its analogues. Bioorg Med Chem Lett 22:1023-1026.

Bikbulatov R V, Yan F, Roth B L, Zjawiony J K (2007) Convenient synthesis and in vitro pharmacological activity of 2-thioanalogs of salvinorins A and B. Bioorg Med Chem Lett 17:2229-2232.

Braida D, Donzelli A, Martucci R, Capurro V, Sala M (2011) Learning and memory impairment induced by salvinorin A, the principal ingredient of Salvia divinorum, in wistar rats. Int J Toxicol 30:650-661.

Braveman F R, Scanove B M, Blessing M E, Wong C A (2013) Obstetrical Anesthesia. In: Clinical Anesthesia, 7th ed. (Barash P G, Cullen B F, Stoelting R K, Cahalan M K, Ortega R, eds), pp 1144-1177. Philadelphia: Lippincott Williams & Wilkins.

Butelman E R, Harris T J, Kreek M J (2004) The plant-derived hallucinogen, salvinorin A, produces kappa-opioid agonist-like discriminative effects in rhesus monkeys. Psychopharmacology (Berl) 172:220-224.

Butelman E R, Prisinzano T E, Deng H, Rus S, Kreek M J (2009) Unconditioned behavioral effects of the powerful kappa-opioid hallucinogen salvinorin A in nonhuman primates: fast onset and entry into cerebrospinal fluid. J Pharmacol Exp Ther 328:588-597.

Carlezon W A, Béguin C, DiNieri J A, Baumann M H, Richards M R, Todtenkopf M S, Rothman R B, Ma Z, Lee D Y-W, Cohen B M (2006) Depressive-like effects of the kappa-opioid receptor agonist salvinorin A on behavior and neurochemistry in rats. J Pharmacol Exp Ther 316:440-447.

Chartoff E H, Potter D, Damez-Werno D, Cohen B M, Carlezon W A (2008) Exposure to the selective kappa-opioid receptor agonist salvinorin A modulates the behavioral and molecular effects of cocaine in rats. Neuropsychopharmacol Off Publ Am Coll Neuropsychopharmacol 33:2676-2687.

Crowder C M, Palanca B J, Evers A S (2013) Mechanisms of Anesthesia and Consciousness. In: Clinical Anesthesia, 7th ed. (Barash P G, Cullen B F, Stoelting R K, Cahalan M K, Stock M C, Ortega R, eds), pp 107-129. Philadelphia: Lippincott Williams & Wilkins.

Cunningham C W, Rothman R B, Prisinzano T E (2011) Neuropharmacology of the naturally occurring kappa-opioid hallucinogen salvinorin A. Pharmacol Rev 63:316-347.

Dahan A, Niesters M, Olofsen E, Smith T, Overdyk F (2013) Opioids. In: Clinical Anesthesia, 7th ed. (Barash P G, Cullen B F, Stoelting R K, Cahalan M K, eds), pp 501-522. Philadelphia: Lippincott Williams & Wilkins.

Ebert T J, Lindenbaum L (2013) Inhaled Anesthetics. In: Clinical Anesthesia, 7th ed. (Barash P G, Cullen B F, Stoelting R K, Cahalan M K, Ortega R, eds), pp 447-477. Philadelphia: Lippincott Williams & Wilkins.

Eger E I, Saidman L J, Brandstater B (1965) Minimum alveolar anesthetic concentration: a standard of anesthetic potency. Anesthesiology 26:756-763.

Fantegrossi W E, Kugle K M, Valdes L J, Koreeda M, Woods J H (2005) Kappa-opioid receptor-mediated effects of the plant-derived hallucinogen, salvinorin A, on inverted screen performance in the mouse. Behav Pharmaco116:627-633.

Fichna J, Lewellyn K, Yan F, Roth B L, Zjawiony J K (2011) Synthesis and biological evaluation of new salvinorin A analogues incorporating natural amino acids. Bioorg Med Chem Lett 21:160-163.

Fish R, Danneman P J, Brown M, Karas A (2011) Anesthesia and Analgesia in Laboratory Animals Academic Press.

Gustafsson L L, Ebling W F, Osaki E, Stanski D R (1996) Quantitation of depth of thiopental anesthesia in the rat. Anesthesiology 84:415-427.

Harding W W, Tidgewell K, Byrd N, Cobb H, Dersch C M, Butelman E R, Rothman R B, Prisinzano T E (2005) Neoclerodane diterpenes as a novel scaffold for mu opioid receptor ligands. J Med Chem 48:4765-4771.

Harding W W, Schmidt M, Tidgewell K, Kannan P, Holden K G, Gilmour B, Navarro H, Rothman R B, Prisinzano T E (2006) Synthetic studies of neoclerodane diterpenes from Salvia divinorum: semisynthesis of salvinicins A and B and other chemical transformations of salvinorin A. J Nat Prod 69:107-112.

Hata T M, Hata J S (2013) Preoperative Patient Assessment and Management. In: Clinical Anesthesia, 7th ed. (Barash P G, Cullen B F, Stoelting R K, Cahalan M K, Stock M C, Ortega R, eds). Philadelphia: Lippincott Williams & Wilkins.

Hirasawa S, Cho M, Brust T F, Roach J J, Bohn L M, Shenvi R A (2018) O6C-20-nor-salvinorin A is a stable and potent KOR agonist. Bioorg Med Chem Lett pii: 50960-894X(18)30066-0. doi: 10.1016/j.bmcl.2018.01.055. [Epub ahead of print]

Killinger B A, Peet M M, Baker L E (2010) Salvinorin A fails to substitute for the discriminative stimulus effects of LSD or ketamine in Sprague-Dawley rats. Pharmacol Biochem Behav 96:260-265.

Krystal J H, Karper L P, Seibyl J P, Freeman G K, Delaney R, Bremner J D, Heninger G R, Bowers M B, Charney D S (1994) Subanesthetic effects of the noncompetitive NMDA antagonist, ketamine, in humans. Psychotomimetic, perceptual, cognitive, and neuroendocrine responses. Arch Gen Psychiatry 51:199-214.

Kurczab R, Canale V, Zajdel P, Bojarski A J (2016). An Algorithm to Identify Target-Selective Ligands—A Case Study of 5-HT7/5-HT1A Receptor Selectivity. PLoS One. 2016 Jun. 7; 11(6):e0156986.

Lee D Y W, He M, Kondaveti L, Liu-Chen L-Y, Ma Z, Wang Y, Chen Y, Li J-G, Beguin C, Carlezon W A, Cohen B (2005) Synthesis and in vitro pharmacological studies of C(4) modified salvinorin A analogues. Bioorg Med Chem Lett 15:4169-4173.

Lee D Y W, Deng G, Ma Z, Xu W, Yang L, Liu J, Dai R, Liu-Chen L-Y (2015) Synthesis and biological evaluation of 2-alkyl-2-methoxymethyl-salvinorin ethers as selective κ-opioid receptor agonists. Bioorg Med Chem Lett 25:4689-4692.

Li R, Morris-Natschke S L, Lee K H (2016) Clerodane diterpenes: sources, structures and biological activities. Nat Prod Rep 33(10):1166-226.

Macres S M, Moore P G, Fishman S M (2013) Acute Pain Management. In: Clinical Anesthesia, 7th ed., pp 1611-1644. Philadelphia: Lippincott Williams & Wilkins.

Maqueda A E, Valle M, Addy P H, Antonijoan R M, Puntes M, Coimbra J, Ballester M R, Garrido M, Gonzalez M, Claramunt J, Barker S, Johnson M W, Griffiths R R, Riba J (2015) Salvinorin A Induces Intense Dissociative Effects, Blocking External Sensory Perception and Modulating Interoception and Sense of Body Ownership in Humans. Int J Neuropsychopharmacol Off Sci J Coll Int Neuropsychopharmacol CINP 18:pii: pyv065.

Maqueda A E, Valle M, Addy P H, Antonijoan R M, Puntes M, Coimbra J, Ballester M R, Garrido M, Gonzalez M, Claramunt J, Barker S, Lomnicka I, Waguespack M, Johnson M W, Griffiths R R, Riba J (2016) Naltrexone but Not Ketanserin Antagonizes the Subjective, Cardiovascular, and Neuroendocrine Effects of Salvinorin A in Humans. Int J Neuropsychopharmacol 19:pii:pyw016.

McCurdy C R, Sufka K J, Smith G H, Warnick J E, Nieto M J (2006) Antinociceptive profile of salvinorin A, a structurally unique kappa opioid receptor agonist. Pharmacol Biochem Behav 83:109-113.

McGovern D L, Mosier P D, Roth B L, Westkaemper R B (2010) CoMFA analyses of C-2 position salvinorin A analogs at the kappa-opioid receptor provides insights into epimer selectivity. J Mol Graph Model 28:612-625.

Muir W W, Hubbell J A E, Bednarski R M, Lerche P (2013) Handbook of Veterinary Anesthesia, 5th ed. Elsevier.

Munro T A, Rizzacasa M A (2003) Salvinorins D-F, new neoclerodane diterpenoids from Salvia divinorum, and an improved method for the isolation of salvinorin A. J Nat Prod 66:703-705.

Munro T A, Rizzacasa M A, Roth B L, Toth B A, Yan F (2005) Studies toward the pharmacophore of salvinorin A, a potent kappa opioid receptor agonist. J Med Chem 48:345-348.

Munro T A, Duncan K K, Xu W, Wang Y, Liu-Chen L-Y, Carlezon W A, Cohen B M, Beguin C (2008) Standard protecting groups create potent and selective kappa opioids: salvinorin B alkoxymethyl ethers. Bioorg Med Chem 16:1279-1286.

Nozawa M, Suka Y, Hoshi T, Suzuki T, Hagiwara H (2008) Total synthesis of the hallucinogenic neoclerodane diterpenoid salvinorin A. Org Lett 10:1365-1368.

Ortega A, Blount J F, Manchand P S (1982) Salvinorin, a new trans-neoclerodane diterpene from Salvia divinorum (Labiatae). J Chem Soc [Perkin 1] :2505-2508.

Placzek M S, Van de Bittner G C, Wey H-Y, Lukas S E, Hooker J M (2015) Immediate and Persistent Effects of Salvinorin A on the Kappa Opioid Receptor in Rodents, Monitored In Vivo with PET. Neuropsychopharmacol Off Publ Am Coll Neuropsychopharmaco140:2865-2872.

Polepally P R, White K, Vardy E, Roth B L, Ferreira D, Zjawiony J K (2013) Kappa-opioid receptor-selective dicarboxylic ester-derived salvinorin A ligands. Bioorg Med Chem Lett 23:2860-2862.

Polepally P R, Huben K, Vardy E, Setola V, Mosier P D, Roth B L, Zjawiony J K (2014) Michael acceptor approach to the design of new salvinorin A-based high affinity ligands for the kappa-opioid receptor. Eur J Med Chem 85:818-829.

Prisinzano and Rothman (2008). Salvinorin A Analogs as Probes in Opioid Pharmacology. Chem Rev 108(5):1732-43.

Ray T S (2010) Psychedelics and the human receptorome. PloS One 5:e9019.

Riley A P, Day V W, Navarro H A, Prisinzano T E (2013) Palladium-Catalyzed Transformations of Salvinorin A, a Neoclerodane Diterpene from Salvia divinorum. Org Lett 15:5936-5939.

Riley A P, Groer C E, Young D, Ewald A W, Kivell B M, Prisinzano T E (2014) Synthesis and κ-opioid Receptor Activity of Furan-Substituted Salvinorin A Analogues. J Med Chem 57:10464-10475.

Roach J J, Sasano Y, Schmid C L, Zaidi S, Katritch V (2017) Dynamic Strategic Bond Analysis Yields a Ten-Step Synthesis of 20-nor-Salvinorin A, a Potent κ-OR Agonist. ACS Cent Sci 3(12):1329-1336.

Roth B L, Baner K, Westkaemper R, Siebert D, Rice K C, Steinberg S, Ernsberger P, Rothman R B (2002) Salvinorin A: a potent naturally occurring nonnitrogenous kappa opioid selective agonist. Proc Natl Acad Sci USA 99:11934-11939.

Sherwood A M, Crowley R S, Paton K F, Biggerstaff A, Neuenswander B, Day V W, Kivell B M, Prisinzano T E (2017a) Addressing Structural Flexibility at the A-Ring on Salvinorin A: Discovery of a Potent Kappa-Opioid Agonist with Enhanced Metabolic Stability. J Med Chem 60:3866-3878.

Sherwood A M, Williamson S E, Crowley R S, Abbott L M, Day V W, Prisinzano T E (2017b) Modular Approach to pseudo-Neoclerodanes as Designer κ-opioid Ligands. Org Lett 19: 5414-5417.

Siebert. D (2012) The Salvia divinorum FAQ. Available at: http://www.sagewisdom.org/faq.html#Section%2011.

Stewart D J, Fahmy H, Roth B L, Yan F, Zjawiony J K (2006) Bioisosteric modification of salvinorin A, a potent and selective kappa-opioid receptor agonist. Arzneimittelforschung 56:269-275.

Teksin Z S, Lee I J, Nemieboka N N, Othman A A, Upreti V V, Hassan H E, Syed S S, Prisinzano T E, Eddington N D (2009) Evaluation of the transport, in vitro metabolism and pharmacokinetics of Salvinorin A, a potent hallucinogen. Eur J Pharm Biopharm Off J Arbeitsgemeinschaft Pharm Verfahrenstechnik EV 72:471-477.

Tidgewell K, Harding W W, Schmidt M, Holden K G, Murry D J, Prisinzano T E (2004) A facile method for the preparation of deuterium labeled salvinorin A: synthesis of [2,2,2-2H3]-salvinorin A. Bioorg Med Chem Lett 14:5099-5102.

Tidgewell K, Harding W W, Lozama A, Cobb H, Shah K, Kannan P, Dersch C M, Parrish D, Deschamps J R, Rothman R B, Prisinzano T E (2006) Synthesis of salvinorin A analogues as opioid receptor probes. J Nat Prod 69:914-918.

Tidgewell K, Groer C E, Harding W W, Lozama A, Schmidt M, Marquam A, Hiemstra J, Partilla J S, Dersch C M, Rothman R B, Bohn L M, Prisinzano T E (2008) Herkinorin analogues with differential beta-arrestin-2 interactions. J Med Chem 51:2421-2431.

Valdes L J, Butler W M, Hatfield G M, Paul A G, Koreeda M (1984) Divinorin A, a psychotropic terpenoid, and divinorin B from the hallucinogenic Mexican mint, Salvia divinorum. J Org Chem 49:4716-4720.

Waldhoer M, Bartlett S E, Whistler J L (2004) Opioid receptors. Annu Rev Biochem 73:953-990.

Wang Y, Chen Y, Xu W, Lee D Y W, Ma Z, Rawls S M, Cowan A, Liu-Chen L-Y (2008) 2-Methoxymethyl-salvinorin B is a potent kappa opioid receptor agonist with longer lasting action in vivo than salvinorin A. J Pharmacol Exp Ther 324:1073-1083.

White P F, Eng M R (2013) Intravenous Anesthetics. In: Clinical Anesthesia, 7th ed. (Barash P G, Cullen B F, Stoelting R K, Cahalan M K, Stock M C, Ortega R, eds), pp 478-500. Philadelphia: Lippincott Williams & Wilkins.

White K L, Scopton A P, Rives M-L, Bikbulatov R V, Polepally P R, Brown P J, Kenakin T, Javitch J A, Zjawiony J K, Roth B L (2014) Identification of novel functionally selective κ-opioid receptor scaffolds. Mol Pharmacol 85:83-90.

White K L, Robinson J E, Zhu H, DiBerto J F, Polepally P R, Zjawiony J K, Nichols D E, Malanga C J, Roth B L (2015) The G Protein-Biased κ-opioid Receptor Agonist RB-64 Is Analgesic with a Unique Spectrum of Activities In Vivo. J Pharmacol Exp Ther 352:98-109.

Willmore-Fordham C B, Krall D M, McCurdy C R, Kinder D H (2007) The hallucinogen derived from Salvia divinorum, salvinorin A, has kappa-opioid agonist discriminative stimulus effects in rats. Neuropharmacology 53:481-486.

Yan F, Bikbulatov R V, Mocanu V, Dicheva N, Parker C E, Wetsel W C, Mosier P D, Westkaemper R B, Allen J A, Zjawiony J K, Roth B L (2009) Structure-based design, synthesis, and biochemical and pharmacological characterization of novel salvinorin A analogues as active state probes of the kappa-opioid receptor. Biochemistry (Mosc) 48:6898-6908.

1. A combination product comprising:
(i) one or more selective κ-opioid receptor agonists, wherein the selective κ-opioid receptor agonist is a compound described by the following formula (I): 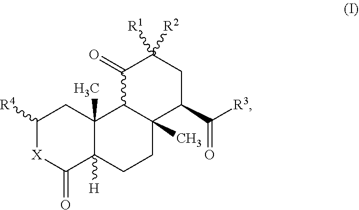
wherein R1, R2, R3 and R4 are selected, independently, from Table 1 and X is C or O, or
R3 and R4 are selected, independently, from Table 1, X is C or O, and R1 and R2 form a 3-5 membered alkyl ring which may be substituted with O and comprises at least one heteroatom which is an O; or
the selective κ-opioid receptor agonist is a compound described by the following formula (II): 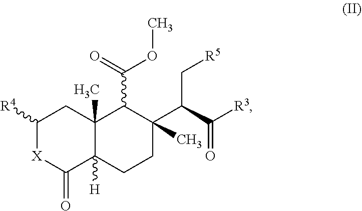
wherein:
R3 and R4 are selected, independently, from Table 1, X is C or O, and R5 is selected from the group consisting of C═O, CH₂OAc and CH(OMe)₂; and
(ii) one or more benzodiazepines and/or one or more α₂-adrenergic receptor agonists;
for use in the induction and/or maintenance of general anesthesia in a subject or animal.

2. The combination product for use according to claim 1, wherein the selective κ-opioid receptor agonist is selected from Table 2.

3. The combination product for use according to claim 1 or 2 wherein:
(a) the α₂-adrenergic receptor agonist is selected from a group consisting of Medetomidine, Dexmedetomidine, Romifidine, Detomidine, Xylazine, Clonidine, Agmatine, Lofexidine, Tizanidine, Guanfacine, Guanabenz and Mivazerol; and/or
(b) the benzodiazepine is selected from a group consisting of Diazepam, Midazolam, Lorazepam, Zolazepam, Etomidate, Adinazolam, Bentazepam, Bromazepam, Brotizolam, Camazepam, Chlorazepam, Chlordiazepoxide, Cinolazepam, Clobazam, Clonazepam, Clotiazepam, Cloxazolam, Estazolam, Alprazolam, Ethyl loflazepate, Etizolam, Fludiazepam, Flunitrazepam, Flurazepam, Halazepam, Ketazolam, Loprazolam, Lormetazepam, Medazepam, Nitrazepam, Nordiazepam, Oxazepam, Pinazepam, Prazepam, Quazepam, Temazepam, Tofisopam, Triazolam, Flutazolam, Flutoprazepam, Nimetazepam, Mexazolam, and Haloxazolam.

4. The combination product for use according to any one of claims 1 to 3, wherein the combination product is prepared for oral, sublingual, buccal, intranasal, intravenous, intramuscular, intraperitoneal and/or inhalation-mediated administration.

5. The combination product for use according to any one of claims 1 to 4, wherein:
(a) the combination product is a composition; or
(b) the one or more selective κ-opioid receptor agonists, and the one or more α₂-adrenergic receptor agonists and/or one or more benzodiazepines are physically separated.

6. The combination product for use according to any one of claims 1-5, wherein the combination product is administered continuously or discontinuously.

7. The combination product for use according to any one of claims 1-6, wherein the κ-opioid receptor agonist, and the α₂-adrenergic receptor agonist and/or benzodiazepine are administered together or separately.

8. The combination product for use according to any one of claims 1 to 7, wherein the combination product is administered intravenously, intraperitoneally or via inhalation.

9. The combination product for use according to any one of claim 1-6 or 8, wherein the α₂-adrenergic receptor agonist and/or benzodiazepine is administered first and then the κ-opioid receptor agonist is administered.

10. The combination product for use according to any one of claims 1 to 9, wherein the κ-opioid receptor agonist and the benzodiazepine is administered at a mass ratio of at least 6:1 and/or the κ-opioid receptor agonist and the α₂-adrenergic receptor agonist is administered at a mass ratio of at least 120:1.

11. A selective κ-opioid receptor agonist for use in a method of inducing or maintaining a state of general anesthesia in a subject or animal, wherein the selective κ-opioid receptor agonist is co-administered with a α₂-adrenergic receptor agonist and/or a benzodiazepine, and
the selective κ-opioid receptor agonist is a compound described by the following formula (I): 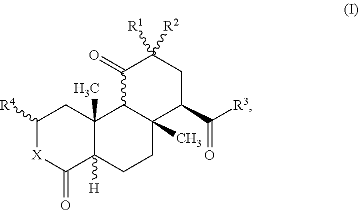
wherein R1, R2, R3 and R4 are selected, independently, from Table 1 and X is C or O, or
R3 and R4 are selected, independently, from Table 1, X is C or O, and R1 and R2 form a 3-5 membered alkyl ring which may be substituted with O and comprises at least one heteroatom which is an O; or
the selective κ-opioid receptor agonist is a compound described by the following formula (II): 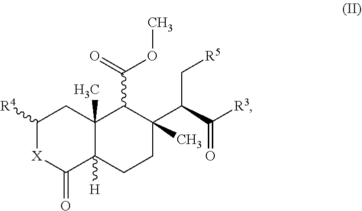
wherein:
R3 and R4 are selected, independently, from Table 1, X is C or O, and R5 is selected from the group consisting of C═O, CH₂OAc and CH(OMe)₂.

12. A α₂-adrenergic receptor agonist and/or a benzodiazepine for use in a method of inducing or maintaining a state of general anesthesia in a subject or animal, wherein the α₂-adrenergic receptor agonist and/or the benzodiazepine is co-administered with a selective κ-opioid receptor agonist, and the selective κ-opioid receptor agonist is a compound described by the following formula (I): 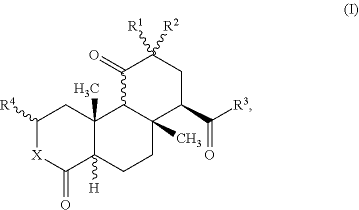
wherein R1, R2, R3 and R4 are selected, independently, from Table 1 and X is C or O, or
R3 and R4 are selected, independently, from Table 1, X is C or O, and R1 and R2 form a 3-5 membered alkyl ring which may be substituted with O and comprises at least one heteroatom which is an O; or
the selective κ-opioid receptor agonist is a compound described by the following formula (II): 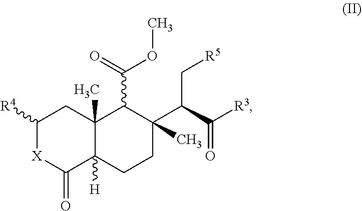
wherein:
R3 and R4 are selected, independently, from Table 1, X is C or O, and R5 is selected from the group consisting of C═O, CH₂OAc and CH(OMe)₂.
